(19) United States
(12) Patent Application Publication
(10) Pub. No.: US 2023/0233139 A1
(43) Pub. Date: Jul. 27, 2023

(54) VAGINAL INSERT DEVICES AND METHODS

(71) Applicant: WATKINS-CONTI PRODUCTS, INC., Edmond, OK (US)

(72) Inventor: Allison CONTI, Edmond, OK (US)

(21) Appl. No.: 17/999,483

(22) PCT Filed: May 21, 2021
(86) PCT No.: PCT/US2021/033732

§ 371 Date: Nov. 21, 2022

Related U.S. Application Data
(60) Provisional application No. 63/028,316, filed on May 21, 2020.
(60) Provisional application No. 63/072,997, filed on Sep. 1, 2020.

(51) Int. Cl. A61B 5/00; A61B 5/07; A61F 6/08; A61F 2/00

(52) U.S. Cl. CPC A61B 5/4337; A61B 5/07; A61F 6/08; A61F 2/005; A61F 2250/0067; A61B 2010/0074

(57) ABSTRACT

A vaginal insert device and method of use for improving and preventing symptoms associated with pelvic organ prolapse and urinary and/or fecal incontinence, administer medication or topical treatment, and/or collect a sample when the device is inserted, or any combination thereof. The vaginal insert device may be cone-shaped, may have a removal portion, may have external ridges, may have internal ribs, may have an open or closed top, or combinations thereof. The device may include cavities, diagnostic devices, and/or microchips.

### CROSS REFERENCE TO RELATED APPLICATIONS

This application claims priority to U.S. Provisional Application No. 63/028,316 filed May 21, 2020 and U.S. Provisional Application No. 63/072,997 filed Sep. 1, 2020, the contents of which are hereby incorporated by reference in their entireties.

This application is related to U.S. Provisional Application No. 62/283,092 filed Aug. 20, 2015, U.S. Provisional Patent Application No. 62/644,340 filed on Mar. 16, 2018, U.S. Provisional Patent Application No. 62/687,119 filed on Jun. 19, 2018, and U.S. Provisional Patent Application No. 62/735,605 filed on Sep. 24, 2018, the contents of which are hereby incorporated by reference in their entireties.

This application is related to U.S. application Ser. No. 15/242,105 filed Aug. 19, 2016, now U.S. Pat. No. 10,188,545, U.S. application Ser. No. 16/224,566 filed Dec. 18, 2018, U.S. application Ser. No. 16/355,638 filed Mar. 15, 2019, International Patent Application No. PCT/US2016/047859 filed Aug. 19, 2016, and International Patent Application No. PCT/US2019/022624 filed Mar. 15, 2019, the contents of which are hereby incorporated by reference in their entireties.

### TECHNICAL FIELD

The present disclosure relates to vaginal insert devices and methods.

### BACKGROUND

Stress Urinary Incontinence (SUI) and Pelvic Organ Prolapse (POP) are growing problems globally that not only cost health care systems large amounts of money, but severely degrade the quality of life of tens of millions of women in the United States alone. SUI in women, is the involuntary leakage of urine due to a weakened pelvic support system and/or pressure on the bladder. This may be caused by aging, genetics, and/or childbirth. The Urology Care Foundation estimates that one of every three women will experience SUI at some point in their lifetime. There are a few types of urinary incontinence including stress incontinence, urge incontinence and mixed incontinence. All are mainly due to connective tissue laxity or damage in the vagina or supportive ligaments.

Further, medication, ointments or topical substances may need to be applied to inner vaginal tissue to treat infections, disease, or alleviate discomfort. However, the substances may have to be re-applied and may quickly dissipate when applied directly to the vaginal tissue. It may also be difficult to apply the substance to the necessary area within the vagina. The medications may also leak from the vaginal canal throughout the day creating discomfort, wetness, and inconvenience for the user.

Although surgical solutions may succeed in ameliorating symptoms associated with SUI and POP, surgery is not without risks and complications and may even leave the user in a worse situation than before treatment. Such surgical methods include transvaginal mesh, which may result in mesh erosion through the vagina, pain, infection, bleeding, discomfort during intercourse, organ perforation, urinary problems, recurrent prolapse, neuro-muscular problems, vaginal scarring/shrinkage & emotional problems. Most surgical complications require intervention including medical, additional surgical treatment and hospitalization. Pessaries are also identified for management of female POP and SUI. Pessaries have had fewer complications and side effects. However, these devices are traditionally placed in the vagina for an extended period of time which may result in discomfort and/or sometime infection. Furthermore, pessaries have been difficult for the user to insert and remove which may require regular office visits with a physician for years. Difficulty with self-removal and insertion of the pessary, having the pessary fall out during defecation, and lack of comfort and convenience may be limiting widespread use of these devices. Over-the-counter treatment options are not user friendly. What is needed is a user-focused solution to these problems.

Additionally, the testing of various medical conditions associated with the reproductive system, including, for example, sexually transmitted diseases (STDs), infections, abnormal cells, temperature, etc., generally involves the collection of a test sample, such as, e.g., a blood, urine, tissue, or discharge sample, from a patient by a medical professional. This collected sample is thereafter tested by an outside laboratory and the results are provided to the patient.

While a few over-the-counter test kits are available for the detection of some types of STDs and/or infections, these over-the-counter testing options are generally not user friendly. What is needed is a user-focused solution to collect a test sample (e.g., a vaginal fluid, discharge and/or tissue sample) from a user, while allowing for at-home detection and/or diagnosis of various diseases and/or medical conditions from this sample without a visit to a doctor or submission of the sample to a laboratory.

### BRIEF SUMMARY

Additional features and advantages of the disclosure will be set forth in the description which follows, and in part will be obvious from the description, or can be learned by practice of the herein disclosed principles. The features and advantages of the disclosure can be realized and obtained by the instruments and combinations particularly pointed out in the appended claims. These and other features of the disclosure will become more fully apparent from the following description and appended claims or can be learned by the practice of the principles set forth herein.

In one aspect of the disclosure, a vaginal insert device for improving, managing, treating, preventing, and/or eliminating symptoms associated with pelvic organ prolapse, urinary incontinence, fecal incontinence, or combinations thereof, the vaginal insert device comprising: an upper portion having a cone-shaped body, a rim, and an upper end; a lower portion extending axially from the upper portion having a lower end; and a surface extending across the upper end such that the cone-shaped body is closed, wherein the rim is configured to apply pressure to an organ wall to improve, manage, treat, prevent, and/or eliminate symptoms associated with pelvic organ prolapse, urinary incontinence, fecal incontinence, or combinations thereof.

Further described herein is a vaginal insert device wherein the rim protrudes from the cone-shaped body at the upper end of the upper portion.

Further described herein is a vaginal insert device comprising an opening in the surface, wherein the opening is located in a center of the surface and is aligned axially with the lower portion.

Further described herein is a vaginal insert device wherein the opening allows for equalization of pressure within a vagina.

Further described herein is a vaginal insert device comprising one or more ridges protruding outwardly from the upper portion, the lower portion, or both the upper portion and the lower portion.

Further described herein is a vaginal insert device wherein the cone-shaped body decreases in diameter from a first diameter at the upper end to a second diameter at a junction of the upper portion and the lower portion.

Further described herein is a vaginal insert device wherein the lower portion increases in diameter from the second diameter to a third diameter at the lower end.

Further described herein is a vaginal insert device wherein the organ wall is one of a urethral sphincter, bladder neck, rectal wall, uterine wall, or combinations thereof.

Further described herein is a vaginal insert device wherein the upper portion, lower portion, and surface are formed integrally of the same material.

In another aspect of the disclosure, a vaginal insert device for improving, managing, treating, preventing, and/or eliminating symptoms associated with pelvic organ prolapse, urinary incontinence, fecal incontinence, or combinations thereof, the vaginal insert device comprising: an upper portion having a cone-shaped body, a rim, and an upper end; and a lower portion extending axially from the upper portion having a lower end; wherein the cone-shaped body is formed of a rib that decreases in diameter from a first diameter at the upper end to a second diameter at a junction between the upper portion and the lower portion, and wherein the rim and the rib are configured to apply pressure to an organ wall to improve, manage, treat, prevent, and/or eliminate symptoms associated with pelvic organ prolapse, urinary incontinence, fecal incontinence, or combinations thereof.

Further described herein is a vaginal insert device wherein the lower portion is cylindrical and has one or more ridges protruding outwardly from an outer surface of the lower portion.

Further described herein is a vaginal insert device wherein the rib is configured to selectively or adjustably apply pressure to the organ wall based on an alignment of the rib with the organ wall.

Further described herein is a vaginal insert device wherein the rib includes two or more ribs spaced equidistantly around a circumference of an interior wall of the upper portion near the rim, the two or more ribs meeting at a center point of the upper portion.

Further described herein is a vaginal insert device wherein the rib forms the cone-shaped body.

Further described herein is a vaginal insert device wherein the rib extends between diametrically opposing points on an interior wall of the upper portion, the rib connected to the interior wall at the diametrically opposing points.

Further described herein is a vaginal insert device wherein, in plan view, the rib is cross-shaped, “T” shaped “X” shaped, “Y” shaped, “K” Shaped, “V” shaped, star shaped, triangular, or pentagonal.

Further described herein is a vaginal insert device wherein in a first position, the rib is aligned with the organ wall and a first pressure is applied to the organ wall and in a second position, the rib is not aligned with the organ wall and a second pressure is applied to the organ wall, the first pressure being greater than the second pressure.

Further described herein is a vaginal insert device wherein the rib forms the cone-shaped body of the upper portion.

Further described herein is a vaginal insert device wherein, in plan view, the rib is a cross-shaped rib having four ends, and wherein each of the four ends meets an interior wall of the upper portion.

Further described herein is a vaginal insert device comprising: an upper portion having a cone-shaped body and an upper end; and a lower portion extending axially from the upper portion having a lower end; wherein the cone-shaped body decreases in diameter from a first diameter at the upper end to a second diameter at the lower portion.

Further described herein is a vaginal insert device further comprising, an outer surface facing vaginal tissue and an inner surface opposite the outer surface.

Further described herein is a vaginal insert device wherein a cavity is arranged between the outer surface and the inner surface.

Further described herein is a vaginal insert device wherein the outer surface is perforated. The perforations may contain a substance such as medication, as described in more detail herein. As described herein, the medication can include treatment of various ailments and/or symptoms thereof, including sexually transmitted diseases or the symptoms thereof.

Further described herein is a vaginal insert device wherein the inner surface not perforated.

Further described herein is a vaginal insert device wherein the outer surface of the cavity is more permeable than the inner surface of the cavity.

Further described herein is a vaginal insert device wherein the cavity is configured to contain a substance.

Further described herein is a vaginal insert device wherein the substance comprises one or more of a topical treatment or a medication.

Further described herein is a vaginal insert device wherein the substance treats or abates one or more of sexually transmitted diseases, yeast infections, and viral or bacterial infections.

Further described herein is a vaginal insert device wherein the substance includes afterbirth medications.

Further described herein is a vaginal insert device wherein the substance includes an agent that provides one or more of birth control, hormone replacement therapy, and cancer medicine.

Further described herein is a vaginal insert device wherein the cone-shaped body is formed of a rib that decreases in diameter from a first diameter at the upper end to a second diameter at a junction between the upper portion and the lower portion, and wherein the rim and the rib are configured to apply pressure to an organ wall to improve, manage, treat, prevent, and/or eliminate symptoms associated with pelvic organ prolapse, urinary incontinence, fecal incontinence, or combinations thereof.

Further described herein is a vaginal insert device wherein the rim and the rib are configured to apply pressure to an organ wall to improve, manage, treat, prevent, and/or eliminate symptoms associated with pelvic organ prolapse, urinary incontinence, fecal incontinence, or combinations thereof.

Further described herein is a vaginal insert device wherein the upper portion is dipped and/or sprayed into a substance.

Further described herein is a vaginal insert device wherein the substance has properties that treat yeast infections.

Further described herein is a vaginal insert device wherein the substance treats yeast infections within 12 hours.

Further described herein is a vaginal insert device having a body configured for placement in a vagina, comprising a coating disposed on at least a portion of the body, the coating comprising a medication.

Further described herein is a vaginal insert device wherein the medication treats or abates one or more of sexually transmitted diseases, yeast infections, and viral or bacterial infections.

Further described herein is a vaginal insert device wherein the medication includes afterbirth medications. The device may be used for pain relief either with or without medication. For example, the device may be chilled or frozen. The device may include liquid or gel, for example, to aid in temperature control.

Further described herein is a vaginal insert device wherein the medication includes an agent that provides one or more of birth control, hormone replacement therapy, and cancer medicine.

Further described herein is a vaginal insert device wherein the medication is adherable to the body in a solid, liquid, powder, or gel form.

Further described herein is a vaginal insert device wherein the medication is absorbable through in vagina.

Further described herein is a vaginal insert device further comprising ridges on an exterior of the body. The ridges may or may not contain medication for treatment of various issues as well as may or may not include gel or liquid or powder for pain relief. It may be heated or cooled for relief of vaginal scarring or tearing due to childbirth. This temperature feature may be used alone or in combination with various treatments and/or a steroid, for example.

Further, the device may be used with urethral sphincter injections to help exercise muscles.

Further described herein is a vaginal insert device wherein the body is further configured to remain in vagina until an extracting force is applied.

Further described herein is a vaginal insert device wherein a rib of the upper most portion is between 37.75 mm and 38.25 mm in diameter.

Further described herein is a vaginal insert device wherein the lower portion contains ridges.

Further described herein is a vaginal insert device wherein the vaginal insert device is between 57.15 mm and 57.65 mm long.

Further described herein is a vaginal insert device wherein the lower portion is between 10.75 mm and 11.25 mm long.

Further described herein is a vaginal insert device wherein the lower portion is between 6.75 mm and 7.25 mm wide.

Further described herein is a vaginal insert device wherein the lower portion is between 4.25 mm and 4.75 mm thick.

Further described herein is a vaginal insert device wherein the ridges are spaced between 2.25 mm and 2.75 mm apart.

Further described herein is a vaginal insert device wherein a rib of the upper most portion is between 43.75 mm and 44.25 mm in diameter.

Further described herein is a vaginal insert device wherein the vaginal insert device is between 58.52 mm and 59.02 mm long.

Further described herein is a vaginal insert device wherein the lower portion is between 12.40 mm and 12.90 mm long.

Further described herein is a vaginal insert device wherein the lower portion is between 6.40 mm and 6.80 mm wide.

Further described herein is a vaginal insert device wherein the lower portion is between 4.93 mm and 5.43 mm thick.

Further described herein is a vaginal insert device wherein the ridges are spaced between 2.63 mm and 3.13 mm apart.

Further described herein is a method of using a vaginal insert device comprising inserting the vaginal insert device into a vagina and delivering medication from the vaginal insert device to tissue inside the vagina.

Further described herein is a vaginal insert device for administering a medication or topical treatment to a vaginal tissue. The vaginal insert device may include an upper portion having a cone-shaped body, a rim, and an upper end; and a lower portion extending axially from the upper portion having a lower end, wherein at least one of the upper portion and the lower portion is configured to include at least one of a medication and a topical treatment for the vaginal tissue. The vaginal insert device upper portion may include a cavity for housing at least one of the medication and the topical treatment. The vaginal insert device upper portion may include perforations configured for administering the medication or topical treatment. The vaginal insert device perforations may be configured to contain the medication or topical treatment. The vaginal insert device may include a coating configured to contain the medication or topical treatment.

In one aspect of the disclosure, a vaginal insert device for collecting a sample is provided. The vaginal insert device comprises an upper portion having a cone-shaped body, a rim, and an open, upper end; a lower portion extending from the upper portion and having a closed, lower end; and a hollow interior, wherein the hollow interior is configured to collect the sample from a vagina.

According to one aspect, the sample is at least one of vaginal fluid, discharge, and a tissue sample.

Further described herein is a vaginal insert device further comprising one or more ridges protruding outwardly from the upper portion, the lower portion, or both the upper portion and the lower portion.

Further described herein is a vaginal insert device wherein the cone-shaped body decreases in diameter from a first diameter at the upper end to a second diameter at a junction of the upper portion and the lower portion.

Further described herein is a vaginal insert device wherein the lower portion increases in diameter from the second diameter to a third diameter at the lower end.

Further described herein is a vaginal insert device comprising an indicator strip for diagnosing and/or detecting an infection and/or medical condition.

Further described herein is a vaginal insert device comprising a microchip for diagnosing and/or detecting an infection and/or medical condition.

Further described herein is a vaginal insert device comprising a texture provided along the exterior of the upper portion, the lower portion, or both the upper portion and the lower portion.

Further described herein is a vaginal insert device wherein the upper portion, lower portion, and surface are formed integrally of the same material.

Further described herein is a vaginal insert device wherein the upper portion has one or more ridges protruding outwardly from an outer surface of the upper portion.

Further described herein is a vaginal insert device wherein the lower portion has one or more ridges protruding outwardly from an outer surface of the lower portion.

Further described herein is a vaginal insert device for collecting a sample comprising: an upper portion having a cone-shaped body and an upper end; and a lower portion extending axially from the upper portion and having a lower end; wherein the cone-shaped body includes at least one texture on an exterior of the body that is configured to collect a tissue sample from a vagina.

Further described herein is a vaginal insert device further comprising an outer surface facing vaginal tissue and an inner surface opposite the outer surface.

Further described herein is a vaginal insert device wherein the outer surface is perforated. The perforations may contain a substance such as medication, as described in more detail herein. As described herein, the medication can include treatment of various ailments and/or symptoms thereof, including sexually transmitted diseases or the symptoms thereof.

Further described herein is a vaginal insert device wherein the substance comprises one or more of a topical treatment or a medication.

Further described herein is a vaginal insert device wherein the substance treats or abates one or more of sexually transmitted diseases, yeast infections, and viral or bacterial infections.

Further described herein is a vaginal insert device wherein the substance includes an agent that provides one or more of birth control, hormone replacement therapy, and cancer medicine.

Further described herein is a vaginal insert device wherein the upper portion is dipped and/or sprayed into a substance.

Further described herein is a vaginal insert device wherein the substance has properties that treat yeast infections.

Further described herein is a vaginal insert device having a body configured for placement in a vagina, comprising a coating disposed on at least a portion of the body, the coating comprising a medication.

Further described herein is a vaginal insert device wherein the medication treats or abates one or more of sexually transmitted diseases, yeast infections, and viral or bacterial infections.

Further described herein is a vaginal insert device wherein the medication is absorbable through in vagina.

Further described herein is a vaginal insert device wherein the body is further configured to remain in vagina until an extracting force is applied.

Further described herein is a method of using a vaginal insert device comprising inserting the vaginal insert device into a vagina and collecting at least one of a vaginal fluid, discharge, and a tissue sample from the vagina.

Further described herein is a method of using a vaginal insert device comprising inserting the vaginal insert device into a vagina, collecting a sample that includes at least one of a vaginal fluid, discharge, and a tissue sample from the vagina, and detecting an infection and/or medical condition using the collected sample.

Further described herein is a method of using a vaginal insert device comprising inserting the vaginal insert device into a vagina and delivering medication from the vaginal insert device to tissue inside the vagina.

In another aspect of the disclosure, a kit is provided. The kit comprises a vaginal insert device that is configured to collect at least one of a vaginal fluid, discharge, and a tissue sample from the vagina, and a container into which the at least one of a vaginal fluid, discharge, and a tissue sample can be released after collection.

Further described herein is a kit further comprising an indicator strip for diagnosing and/or detecting an infection and/or medical condition.

It is to be understood that both the foregoing general description and the following detailed description are examples and explanatory and are not restrictive on the claims set forth in this disclosure.

It is to be understood that both the foregoing general description and the following detailed description are examples and explanatory and are not restrictive on the claims set forth in this disclosure.

### BRIEF DESCRIPTION OF THE DRAWINGS

The foregoing and other features and advantages will be apparent from the following, more particular, description of various exemplary embodiments, as illustrated in the accompanying drawings, wherein like reference numbers generally indicate identical, functionally similar, and/or structurally similar elements.

FIG. 1 illustrates a cross-section of the pelvic region of a female, according to an embodiment of the present disclosure.

FIG. 2 illustrates a cross-section of the pelvic region of a female experiencing incontinence, according to an embodiment of the present disclosure.

FIG. 3 illustrates a cross-section of the pelvic region of a female experiencing bladder prolapse and/or cystocele, according to an embodiment of the present disclosure.

FIG. 4 illustrates a cross-section of the pelvic region of a female experiencing back passage prolapse and/or rectocele, according to an embodiment of the present disclosure.

FIG. 5 illustrates a cross-section of the pelvic region of a female experiencing womb and/or uterine prolapse, according to an embodiment of the present disclosure.

FIG. 6 illustrates a cross-section of the pelvic region of a female with a vaginal insert device provided therein, according to an embodiment of the present disclosure.

FIG. 7 illustrates a cross-section of the pelvic region of a female with a vaginal insert device provided therein, according to an embodiment of the present disclosure.

FIG. 8 illustrates a cross-section of the pelvic region of a female with a vaginal insert device provided therein, according to an embodiment of the present disclosure.

FIG. 9 illustrates a vaginal insert device in a compact position, according to an embodiment of the present disclosure.

FIG. 10 illustrates a method for inserting a vaginal insert device, according to an embodiment of the present disclosure.

FIGS. 11A to 11C illustrate perspective views of a vaginal insert device, according to an embodiment of the present disclosure.

FIGS. 12A to 12C illustrate perspective views of a vaginal insert device, according to an embodiment of the present disclosure.

FIGS. 13A to 13C illustrate perspective views of a vaginal insert device, according to an embodiment of the present disclosure.

FIGS. 14A to 14C illustrate perspective views of a vaginal insert device, according to an embodiment of the present disclosure.

FIGS. 15A to 15C illustrate perspective views of a vaginal insert device, according to an embodiment of the present disclosure.

FIG. 16A illustrates a perspective view of a vaginal insert device, according to an embodiment of the present disclosure.

FIG. 16B illustrates a cross-sectional view of the vaginal insert device of FIG. 16A, taken along a centerline of the vaginal insert device, according to an embodiment of the present disclosure.

FIG. 17 illustrates a cross-sectional view of a vaginal insert device, taken along a centerline of the vaginal insert device, according to an embodiment of the present disclosure.

FIG. 18 illustrates a cross-sectional view of a vaginal insert device, according to an embodiment of the present disclosure.

FIGS. 19A to 19F illustrate views of a vaginal insert device, according to an embodiment of the present disclosure.

FIGS. 20A to 20F illustrate views of a vaginal insert device, according to an embodiment of the present disclosure.

FIG. 21A illustrates a perspective view of a vaginal insert device, according to an embodiment of the present disclosure.

FIG. 21B illustrates a cross-sectional view of the vaginal insert device of FIG. 21A, taken along a centerline of the vaginal insert device, according to an embodiment of the present disclosure.

FIG. 22 illustrates a perspective view of a vaginal insert device, according to an embodiment of the present disclosure.

FIG. 23 illustrates a perspective view of a kit having a vaginal insert device, according to an embodiment of the present disclosure.

### DETAILED DESCRIPTION

Various embodiments are discussed in detail below. While specific embodiments are discussed, this is done for illustration purposes only. A person skilled in the relevant art will recognize that other components and configurations may be used without departing from the spirit and scope of the present disclosure.

The present disclosure describes various embodiments of a vaginal insert device and the method of using the vaginal insert device. The vaginal insert devices of the present disclosure may manage, improve, treat, prevent, and/or eliminate female incontinence, Pelvic Organ Prolapse (POP), or both incontinence and POP. The vaginal insert device of the present disclosure may not require a prescription, may be non-absorbent, over the counter, convenient, comfortable, and/or easy for a user to insert and remove, with no or minimal physician intervention. Such a vaginal insert device may be reusable or may be disposable. Various users are contemplated and users may include patients, consumer, etc.

The present disclosure relates in general to vaginal insert devices and methods. One aspect relates to a vaginal insert and methods for improving and preventing symptoms associated with pelvic organ prolapse and urinary and/or fecal incontinence when the device is inserted. Another aspect relates to a vaginal insert and methods for use with medications as described in more detail below. These devices and methods associated with medications as described herein may be used alone, and/or, in combination with the devices and methods described for improving and preventing symptoms discussed above.

The present disclosure relates in general to vaginal insert devices and methods. One aspect relates to a vaginal insert and methods for collecting a vaginal fluid, discharge and/or tissue sample. Another aspect relates to a vaginal insert and methods for detecting and/or diagnosing various diseases and/or medical conditions. These devices and methods associated with collecting a vaginal fluid, discharge and/or tissue sample as described herein may be used alone, and/or, in combination with the devices and methods described for detecting and/or diagnosing various diseases and/or medical conditions discussed above.

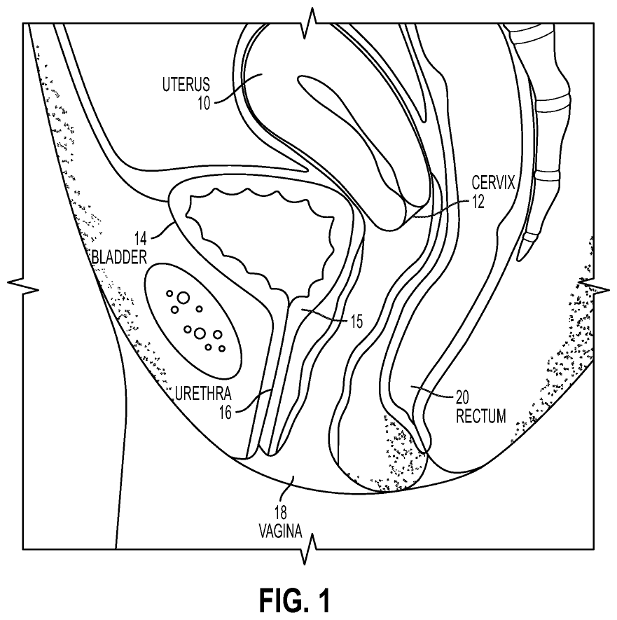

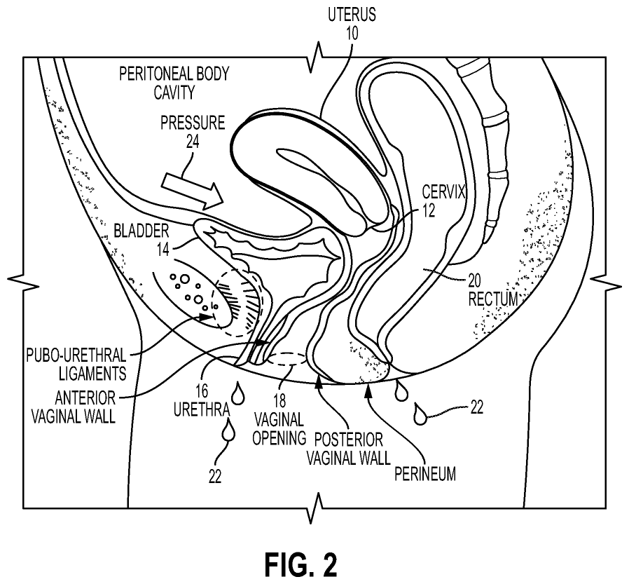

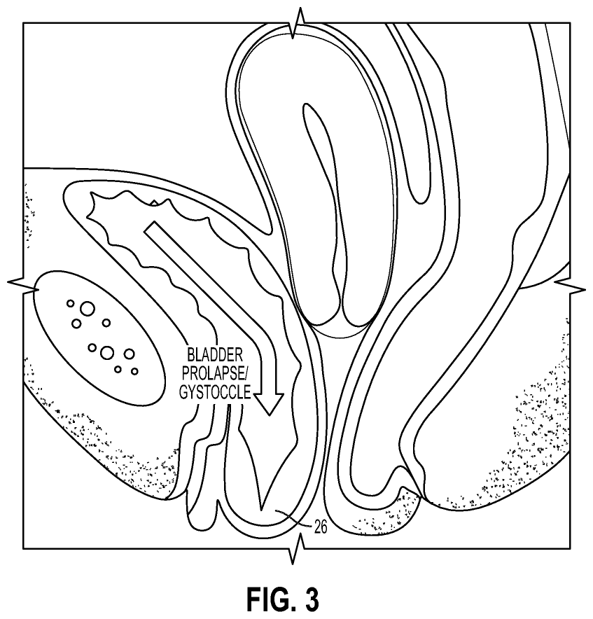

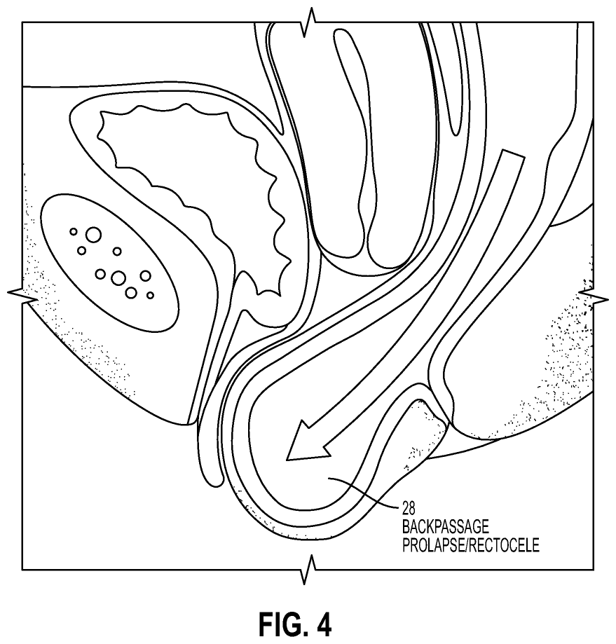

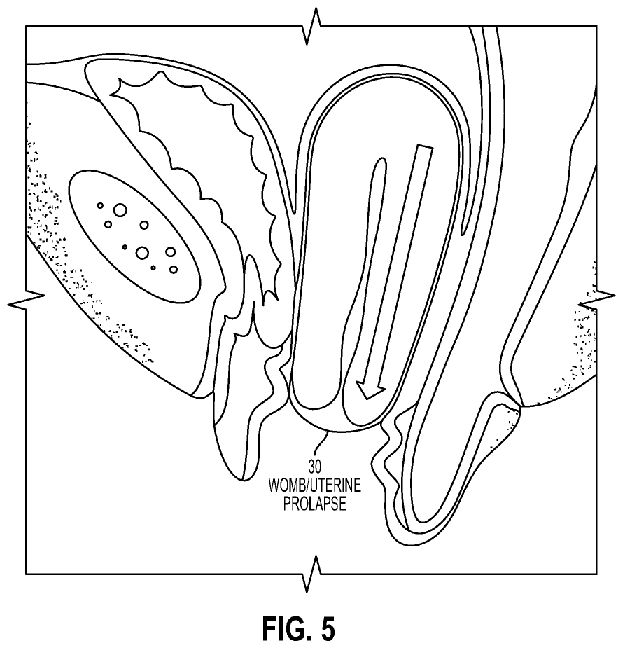

FIGS. 1 and 2 each show a cross-section of the pelvic region of a female anatomy illustrating the uterus 10, cervix 12, bladder 14, bladder neck 15, urethra/urethral sphincter 16, vagina 18, and rectum 20. In FIG. 1, the female anatomy is a normal condition (e.g., a condition not experiencing incontinence and/or prolapse) FIG. 2 illustrates incontinence 22 (e.g., leakage of urine or fecal matter) caused by stress or pressure 24 on the bladder 14 or bowels (e.g., rectum 20). Involuntary leakage of urine or fecal matter may occur during activities such as coughing, laughing, sneezing, lifting or exercise. Connective tissue damage to three zones of the Integral System, which encompasses all three pelvic organs, including the bladder, vagina, and ano-rectum, is the ultimate cause of pelvic organ prolapse (POP) and dysfunction in these organs. FIG. 3 is a cross-section of the pelvic region of a female with a prolapsed bladder 26. FIG. 4 is a cross-section of the pelvic region of a female with a prolapsed back-passage or rectocele 28. FIG. 5 is a cross-section of the pelvic region of a female with a prolapsed uterus 30. POP is commonly due to childbearing but may also be caused simply by genetics and the aging process. The vaginal insert devices of the present disclosure may be inserted into the vagina to manage, improve, treat, prevent, and/or eliminate these conditions.

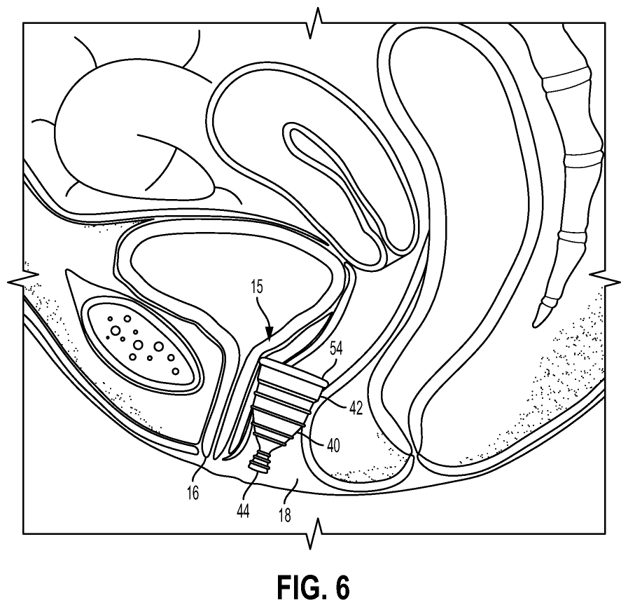

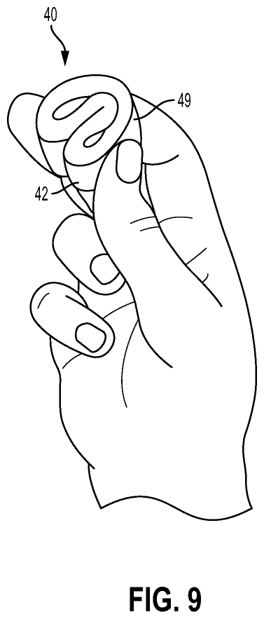

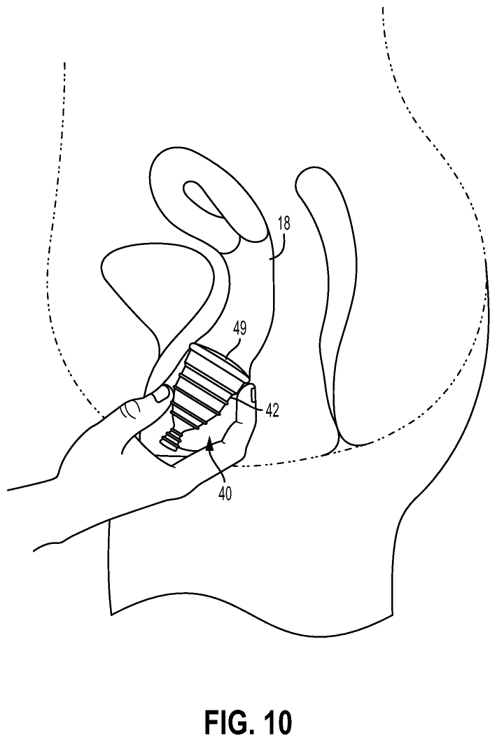

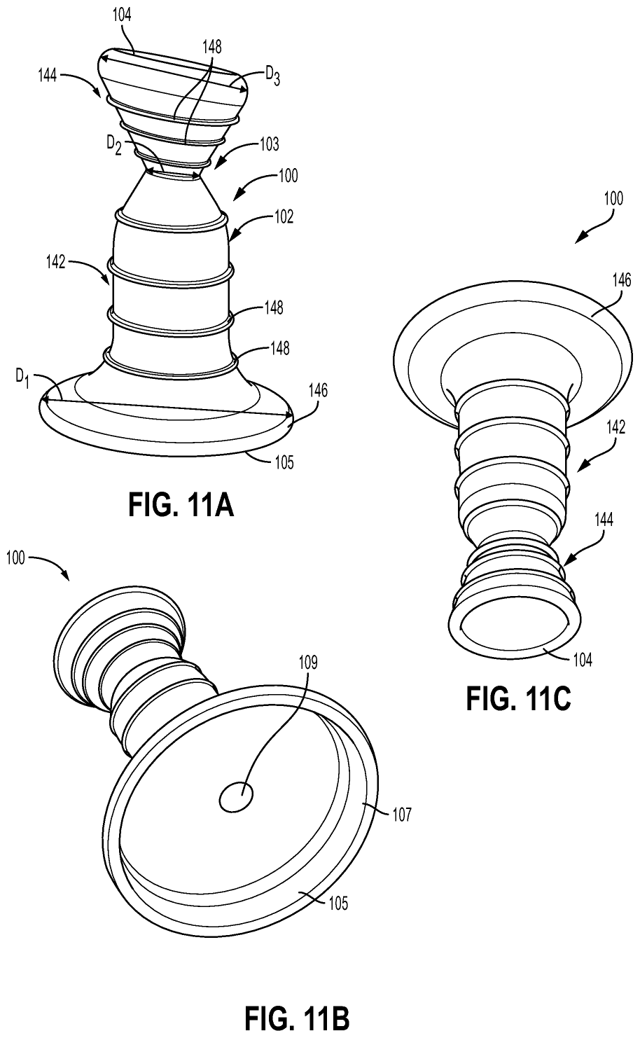

FIG. 6 is a cross-section of the pelvic region of a female illustrating a vaginal insert device 40, according to the present disclosure, inserted in the vagina 18 and applying pressure on the urethral sphincter 16 and/or the bladder neck 15 to manage, improve, prevent, treat, and/or eliminate female urinary incontinence. A user may insert the device 40 according to FIGS. 9 and 10. The device 40 may include an upper portion 42, a lower portion 44, and an upper end 54. Once inserted, the rim (e.g., rim 146 in FIG. 11) of the upper portion 42 may be aligned with an intravaginal wall, such as the urethral sphincter 16 and/or bladder neck 15 to apply pressure thereto. The ridges (e.g., 148 in FIG. 11) may also apply pressure to the intravaginal walls. The pressure applied at the urethral sphincter 16 and/or bladder neck 15 may reduce and/or eliminate urine leakage. Although described with respect to urine leakage, the vaginal insert device 40 may impose pressure to a rectal wall to reduce and/or eliminate fecal matter leakage.

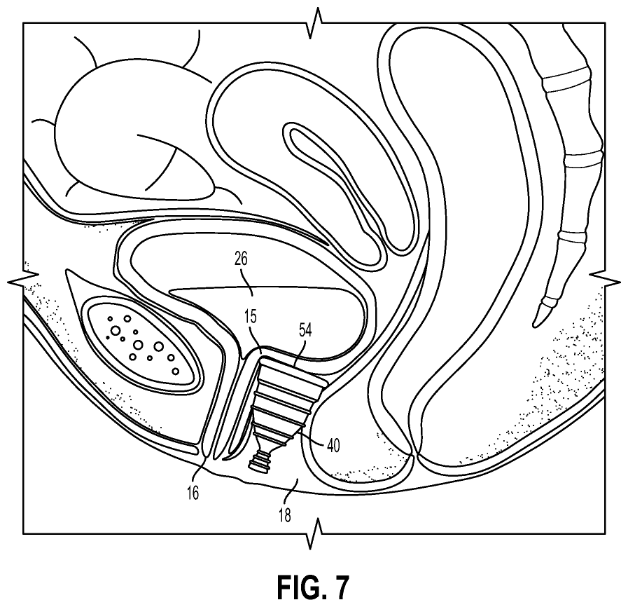

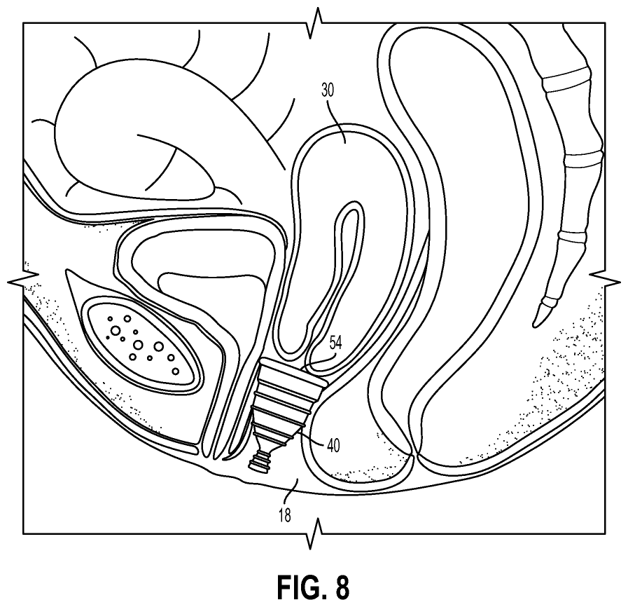

FIGS. 7 and 8 are each a cross-section of the pelvic region of a female illustrating an embodiment of the vaginal insert device 40 inserted in the vagina 18 to manage, improve, prevent, treat, and/or eliminate POP, in addition to applying pressure on the urethral sphincter 16 and/or the bladder neck 15 to manage, improve, or eliminate female urinary incontinence. In particular, in FIG. 7, vaginal insert device 40 is inserted in the vagina 18 to manage, improve, prevent, treat, and/or eliminate a prolapsed bladder 26. In FIG. 7, the upper end 54 may support the bladder 26. The rim and ridges (as described herein) may also apply pressure to an intravaginal wall, such as the urethral sphincter 16 and/or bladder neck 15. The pressure applied at the urethral sphincter 16 and/or bladder neck 15 may reduce or eliminate urine leakage and/or may support the bladder 26. In FIG. 8, vaginal insert device 40 is inserted in the vagina 18 to manage, improve, prevent, treat, and/or eliminate a prolapsed uterus 30. In FIG. 8, the upper end 54 may support the uterus 30. Although described separately, the device 40 may apply pressure to the intravaginal walls (e.g., FIGS. 6 and 7), support the bladder (FIG. 7), support the uterus (FIG. and 8), support and/or apply pressure to a rectal wall, and/or any combination thereof, simultaneously or as the body changes over time (e.g., as the bladder fills).

As illustrated in FIGS. 6-8, the upper end 54 of the upper portion 42 is the innermost portion of the vaginal insert device 40 when inserted into the vagina. As further illustrated in FIGS. 6-8, the lower portion 44 of the vaginal insert device 40 may be accessed from the exterior of the vagina 18 when the vaginal insert device is inserted. The lower portion 44 may assist in insertion, removal, and/or positioning of the vaginal insert device 40 within the vagina. Ridges on the lower portion 44 may provide better grip for removal of the device 40 by a user. The lower portion 44 may also be used to position the device for therapy force, organ support and comfort. The rim and/or the ridges on the upper portion 42 may apply pressure to the organ walls shown in FIGS. 6-8 to manage, improve, prevent, treat, and/or eliminate female incontinence, including urinary incontinence and fecal incontinence, POP, or POP and urinary and/or fecal incontinence, and combinations thereof.

FIGS. 9 and 10 illustrate a method for inserting vaginal insert device 40 into vagina 18. As illustrated in FIG. 9, a user may squeeze the wall 49 of the upper portion 42 to make the upper portion more compact. The more compact shape may allow for easier insertion of the vaginal insert device 40 into the vagina 18. The user may manually squeeze the upper portion 42 between two or more fingers or otherwise within the hand. Alternatively, the user may use a tool, such as the applicator, to assist in squeezing the device 40 for insertion. As illustrated in FIG. 10, the user may then insert the more compact shaped device 40 (as shown in FIG. 9) into the vagina 18. The user may insert the device 40 manually (e.g., with one or both hands) or with a tool (e.g., applicator). Once vaginal insert device 40 is inserted into the vagina 18, the wall 49 of upper portion 42 may expand back to its original shape or near original shape, thus allowing the vaginal insert device 40 to apply pressure to the interior walls of the vagina and internal organs. Although this method of insertion is one example, in a higher durometer silicone or other material, the device does not necessarily have to collapse for insertion.

The vaginal insert devices of the present disclosure may be shaped so that the device is held securely in place in the vagina when inserted, as well as shaped to impose pressure on the intravaginal wall for therapy force and/or to support pelvic organs and/or prevent further pelvic organ displacement. In an example, the vaginal insert devices of the present disclosure are for management of stress urinary incontinence (e.g., the involuntary leakage of urine) during activities such as coughing, laughing, sneezing, lifting and exercise for users over the age of eighteen. The device may be inserted by an adult woman for up to about eight to twelve hours at a time before removal and reinsertion, depending on their comfort level. The device may be and remain in the body for longer periods of time.

Referring to FIGS. 11A to 11C, an exemplary vaginal insert device 100 is shown. The vaginal insert device 100 may be used in the same or similar manner as described with respect to vaginal insert device 40 and FIGS. 6-10. The vaginal insert device 100 may include a body 102. The body 102 may include an upper portion 142 and a lower portion 144. The upper portion 142 may be cone-shaped and/or cup-shaped. The upper portion 142 may be bell-shaped such as shown in FIG. 11A. For example, the upper portion 142 may be include an axially extending portion with a concentrically narrowing top portion, a middle portion with a relatively consistent circular cross-section, and a concentrically flaring lower portion. The upper portion 142 may be hollow within the walls of the upper portion 142. The upper portion 142 may have a first diameter D₁ which decreases to a second diameter D₂. The decreasing in diameter may be gradual. The upper portion 142 may include a changing diameter. Although described herein as cone-shaped, other shapes of the upper portion 42 are contemplated. Such other shapes may include changing diameters such that a first diameter is configured to apply pressure to an intravaginal or organ wall and a second diameter is configured to be smaller than the first diameter and not interact with an intravaginal wall. Such shapes may include trapezoid, spheres, cylinders, cubes, pyramids, hexagonal prisms, etc.

The upper portion 142 may include a rim 146. The rim 146 may protrude from the upper portion 142. The rim 146 may be circular and may surround and protrude from the exterior side of the body 102. The rim 146 may be adjacent to the upper end 105. The rim 146 with the upper portion 142 may form a bell-shaped body 102. The rim 146 may be adjustable in height, location, and/or width. The rim 146 may retract and expand for therapy force and/or storage. The rim 146 may be made from one part or more than one part. The rim 146 may be made from one material, more than one material or a combination of materials. The rim 146 may be configured to apply greater pressure in one area than in another area of the rim 146. For example, the rim 146 may be weighted, either by density, thickness, material, attachments, protrusions, etc., in one portion (e.g., one half of the cross-section of the device). This portion may be configured to apply a greater pressure than the remainder of the rim 146. The remainder of the rim 146 may be constructed similarly to the remainder of the device 100 (e.g., of the same material, density, thickness, etc.). The rim 146 may be tilted, lopsided, or angled to place pressure at different locations in the vaginal canal based on anatomy, type of incontinence, and/or prolapse.

The upper portion 142 may include one or more ridges 148. The one or more ridges 148 may be spaced apart along an outer surface of the upper portion 142. The one or more ridges 148 may by spaced apart from a junction 103 of the upper portion 142 and lower portion 144 to the rim 146. The one or more ridges 148 may be space uniformly and/or randomly spaced along the outer surface of the upper portion 142. The one or more ridges 148 may be semi-permeable to allow liquid, gas, lubrication, and/or medication to pass through. The ridges 148 may be circular rings that surround the exterior side of the body 102. Alternatively, ridges 148 may include any protrusions that extend from the of the body 102, such as studs, knobs, buttons, words, numbers, symbols, logos, other shapes, including polygonal, triangular, separated, randomly separated studs, uniformly spaced studs, or square, or combinations thereof. The ridges 148 may be helical, diagonal, longitudinal, or radially placed on the device 100 (either the upper portion 142 and/or the lower portion 144). The ridges 148 may be the same or different on the upper portion 142 and the lower portion 144.

The upper portion 142 may extend from the junction 103 to an upper end 105. The upper end 105 may be closed. The upper end 105 may include a surface 107. The surface 107 may include an opening 109. However, the opening 109 may be optional. The opening 109 may extend into an interior of the upper portion 142. The upper end 105 may be solid. The opening 109 may be a ventilation opening that allows equalization of pressure through the device when inserted into the vagina. The opening 109 may prevent a suction or seal from being created when the device is inserted. The upper end 105 may be formed of a material that is the same or similar as the body 102.

The upper portion 142 may have a circular transverse cross-section throughout its length. The circular transverse cross-section may reduce in diameter from the upper end 105 (D₁) to the junction 103 (D₂). The reduction in diameter may be a reduction in internal diameter of the upper portion 142, a reduction in the external diameter of the upper portion 142, or a combination thereof. The circumference of the upper portion 142 may decrease from the upper end 105 to the junction 103.

The lower portion 144 may be a stem, removal portion, and/or insertion portion. The lower portion 144 may assist in the insertion, removal, and/or positioning of the vaginal insert device 100. The lower portion 144 may be cone-shaped or cup-shaped. The lower portion 144 may increase in diameter from D₂ to a second diameter D₃. The lower portion 144 may include a lower end 104. The lower end 104 may be open. That is, there may be no surface closing the end 104. The lower portion 144 may hollow within the walls of the lower portion. In some examples, the junction 103 may be open such that a passage is formed from opening 109, through the upper portion 142, through the junction 103, and through the lower end 104.

The lower portion 144 may include one or more ridges 148. The one or more ridges 148 may be spaced apart along an outer surface of the lower portion 144. The one or more ridges 148 may by spaced apart from the junction 103 of the upper portion 142 and lower portion 144 the lower end 104 of the lower portion 144. The one or more ridges 148 may be space uniformly and/or randomly spaced along the outer surface of the lower portion 144. The one or more ridges 148 may be semi-permeable to allow liquid, gas, lubrication, and/or medication to pass through. The one or more ridges 148 may be the same or similar to the one or more ridges 148 on the upper portion 142.

The upper portion 142 and the lower portion 144 may be fabricated as an integral, one-piece device formed of a single material. The upper end 105 may be integral with the upper portion 142 and the lower portion 144. For example, the upper portion 142, the lower portion 144, and/or the upper end 105 may be formed of a medical grade silicone. The upper portion 142, the lower portion 144, and/or the upper end 105 may be injection molded as a single, integral device of a medical grade silicone. Alternatively, the upper portion 142, the lower portion 144, and/or the upper end 105 may be made from more than one part and/or more than one material that may attach and/or detach or expand and retract for therapy, treatment, or sterilization purposes. The upper portion 142 and the lower portion 144 may be permanently or detachably coupled together.

The vaginal insert device 100 may come in different sizes, density, shapes, durability and/or different durometers to accommodate women with differing anatomy, women with changing or fluctuating anatomy, to accommodate different uses of the device, and/or different activities performed while using the device. Furthermore, the dimensions of the various sections and portions of the device 100 may be modified from the multiple embodiments illustrated and disclosed herein. For example, the total height of device 100, diameter at the upper end of the upper portion 142, thickness of the wall of the upper portion 142 of the device 100, diameter of the lower end 104, and/or height of the lower portion 144 may be modified. Modifications made to the dimensions may still retain or may improve on the intended usefulness, effectiveness, and other benefits of the device. The dimensions may be modified in any combination or individually.

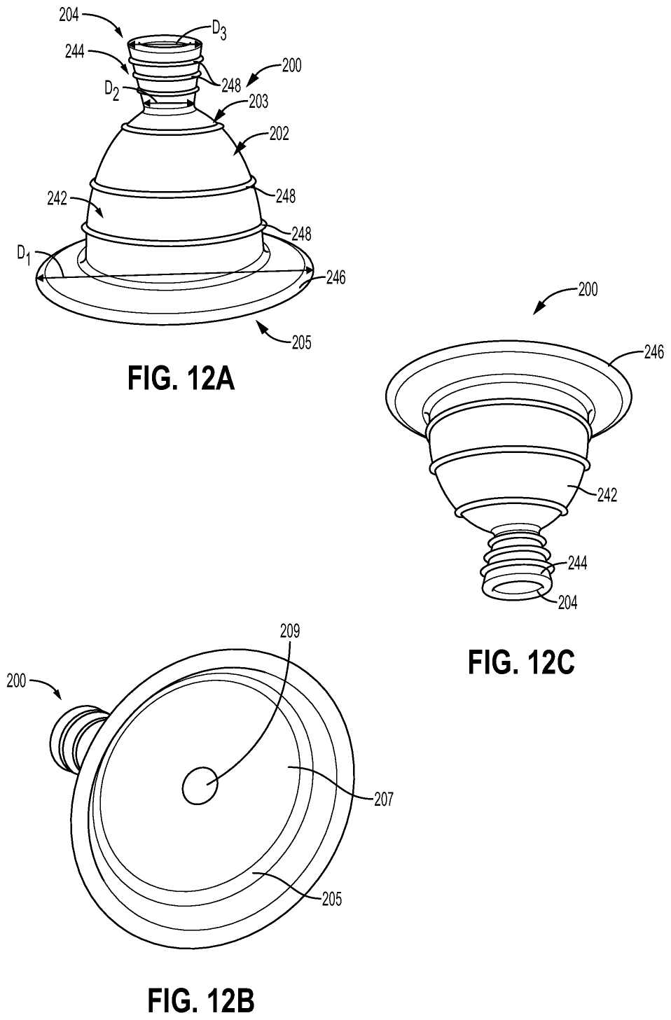

Referring now to FIGS. 12A to 12C, an exemplary vaginal insert device 200 is shown. The vaginal insert device 200 may be similar to the device 100. The vaginal insert device 200 may include any of the modifications, shapes, sizes, materials, dimensions, uses, or any combinations thereof as described with respect to the vaginal insert device 100. Any of the modifications or alternatives of the rim, stem, shape of the upper portion, etc., previously described may be applied separately or in combination to the vaginal insert device 200. The method of insertion and use as described in FIGS. 6-10 may apply to the vaginal insert device 200.

The vaginal insert device 200 may include a body 202. The body 202 may include an upper portion 242 and a lower portion 244. The upper portion 242 may be cone-shaped and/or cup-shaped. The upper portion 242 may be hollow within the walls of the upper portion 242. The upper portion 242 may have a first diameter D₁ which decreases to a second diameter D₂. The decreasing in diameter may be gradual. The upper portion 242 may include a changing diameter. Although described herein as cone-shaped, other shapes of the upper portion 242 are contemplated. Such other shapes may include changing diameters such that a first diameter is configured to apply pressure to an intravaginal or organ wall and a second diameter is configured to be smaller than the first diameter and not interact with an intravaginal wall. Such shapes may include trapezoid, spheres, cylinders, cubes, pyramids, hexagonal prisms, etc.

The upper portion 242 may include a rim 246. The rim 246 may protrude from the upper portion 242. The rim 246 may be circular and may surround and protrude from the exterior side of the body 202. The rim 246 may be adjacent to the upper end 205. The rim 246 may be adjustable in height, location, and/or width. The rim 246 may retract and expand for therapy force and/or storage. The rim 246 may be made from one part or more than one part. The rim 246 may be made from one material, more than one material or a combination of materials. The rim 246 may be configured to apply greater pressure in one area than in another area of the rim 246. For example, the rim 246 may be weighted, either by density, thickness, material, attachments, protrusions, etc., in one portion (e.g., one half of the cross-section of the device). This portion may be configured to apply a greater pressure than the remainder of the rim 246. The remainder of the rim 246 may be constructed similarly to the remainder of the device 200 (e.g., of the same material, density, thickness, etc.). The rim 246 may be tilted, lopsided, or angled to place pressure at different locations in the vaginal canal based on anatomy, type of incontinence, and/or prolapse.

The upper portion 242 may include one or more ridges 248. The one or more ridges 248 may be spaced apart along an outer surface of the upper portion 242. The one or more ridges 248 may by spaced apart from a junction 203 of the upper portion 242 and lower portion 244 to the rim 246. The one or more ridges 248 may be spaced uniformly and/or randomly spaced along the outer surface of the upper portion 242. The one or more ridges 248 may be semi-permeable to allow liquid, gas, lubrication, and/or medication to pass through. The ridges 248 may be circular rings that surround the exterior side of the body 202. Alternatively, ridges 248 may include any protrusions that extend from the of the body 202, such as studs, knobs, buttons, words, numbers, symbols, logos, other shapes, including polygonal, triangular, separated, randomly separated studs, uniformly spaced studs, or square, or combinations thereof. The ridges 248 may be helical, diagonal, longitudinal, or radially placed on the device 200 (either the upper portion 242 and/or the lower portion 244). The ridges 248 may be the same or different on the upper portion 242 and the lower portion 244.

The upper portion 242 may extend from the junction 203 to an upper end 205. The upper end 205 may be closed. The upper end 205 may include a surface 207. The surface 207 may include an opening 209. However, the opening 209 may be optional. The opening 209 may be a ventilation opening that allows equalization of pressure through the device when inserted into the vagina. The opening 209 may prevent a suction or seal from being created when the device is inserted. The opening 209 may extend into an interior of the upper portion 242. The upper end 205 may be solid. The upper end 205 may be formed of a material that is the same or similar as the body 202.

The upper portion 242 may have a circular transverse cross-section throughout its length. The circular transverse cross-section may reduce in diameter from the upper end 205 (D₁) to the junction 203 (D₂). The reduction in diameter may a reduction in internal diameter of the upper portion 242, a reduction in the external diameter of the upper portion 242, or a combination thereof. The circumference of the upper portion 242 may decrease from the upper end 205 to the junction 203.

The lower portion 244 may be a stem, removal portion, and/or insertion portion. The lower portion 244 may assist in the insertion, removal, and/or positioning of the vaginal insert device 200. The lower portion 244 may be cone-shaped or cup-shaped. The lower portion 244 may increase in diameter from D₂ to a second diameter D₃. The lower portion 244 may include a lower end 204. The lower end 204 may be open. That is, there may be no surface closing the end 204. The lower portion 244 may hollow within the walls of the lower portion.

The lower portion 244 may include one or more ridges 248. The one or more ridges 248 may be spaced apart along an outer surface of the lower portion 244. The one or more ridges 248 may by spaced apart from the junction 203 of the upper portion 242 and lower portion 244 the lower end 204 of the lower portion 244. The one or more ridges 248 may be space uniformly and/or randomly spaced along the outer surface of the lower portion 244. The one or more ridges 248 may be semi-permeable to allow liquid, gas, lubrication, and/or medication to pass through. The one or more ridges 248 may be the same or similar to the one or more ridges 248 on the upper portion 242.

The upper portion 242 and the lower portion 244 may be fabricated as an integral, one-piece device formed of a single material. The upper end 205 may be integral with the upper portion 242 and the lower portion 244. For example, the upper portion 242, the lower portion 244, and/or the upper end 205 may be formed of a medical grade silicone. The upper portion 242, the lower portion 244, and/or the upper end 205 may be injection molded as a single, integral device of a medical grade silicone. Alternatively, the upper portion 242, the lower portion 244, and/or the upper end 205 may be made from more than one part and/or more than one material that may attach and/or detach or expand and retract for therapy, treatment, or sterilization purposes. The upper portion 242 and the lower portion 244 may be permanently or detachably coupled together.

The vaginal insert device 200 may come in different sizes, density, shapes, durability and/or different durometers to accommodate women with differing anatomy, women with changing or fluctuating anatomy, to accommodate different uses of the device, and/or different activities performed while using the device. Furthermore, the dimensions of the various sections and portions of the device 200 may be modified from the multiple embodiments illustrated and disclosed herein. For example, the total height of device 200, diameter at the upper end of the upper portion 242, thickness of the wall of the upper portion 242 of the device 200, diameter of the lower end 204, and/or height of the lower portion 244 may be modified. Modifications made to the dimensions may still retain or may improve on the intended usefulness, effectiveness, and other benefits of the device. The dimensions may be modified in any combination or individually.

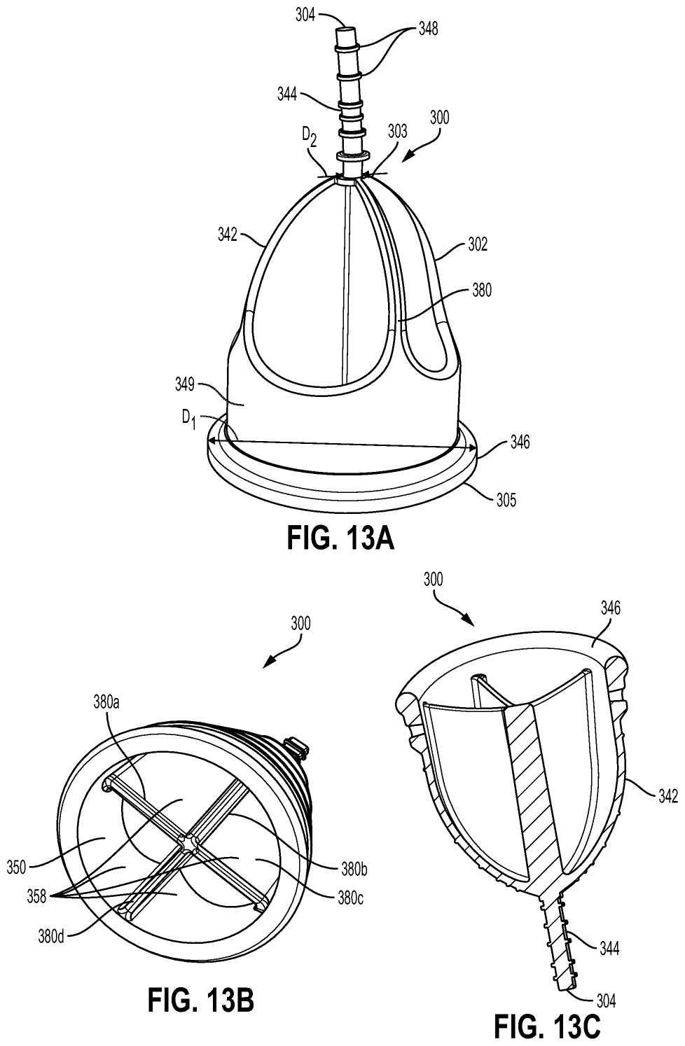

Referring now to FIGS. 13A to -13C, an exemplary vaginal insert device 300 is shown. The vaginal insert device 300 may be similar to the device 100 and/or device 200. The vaginal insert device 300 may include any of the modifications, shapes, sizes, materials, dimensions, uses, or any combinations thereof as described with respect to the vaginal insert device 100 and/or device 200. Any of the modifications or alternatives of the rim, stem, shape of the upper portion, etc., previously described may be applied separately or in combination to the vaginal insert device 300. The method of insertion and use as described in FIGS. 6-10 may apply to the vaginal insert device 300.

The vaginal insert device 300 may include a body 302. The body 302 may include an upper portion 342 and a lower portion 344. The upper portion 342 may include a wall 349, a rim 346, and a member or rib 380 that may be a rib 380. The upper portion 342 may be cone-shaped and/or cup-shaped. The upper portion 342 may be hollow within the wall 349. The upper portion 342 may have a first diameter D₁ which decreases to a second diameter D₂. The decreasing in diameter may be gradual. The upper portion 342 may include a changing diameter. Although described herein as cone-shaped, other shapes of the upper portion 342 are contemplated. Such other shapes may include changing diameters such that a first diameter is configured to apply pressure to an intravaginal or organ wall and a second diameter is configured to be smaller than the first diameter and not interact with an intravaginal wall. Such shapes may include trapezoid, spheres, cylinders, cubes, pyramids, hexagonal prisms, etc.

The upper portion 342 may include the rim 346. The rim 346 may protrude from the wall 349 of the upper portion 342. The rim 346 may be circular and may surround and protrude from the wall 349. The rim 346 may be adjacent to the upper end 305. The rim 346 may be adjustable in height, location, and/or width. The rim 346 may retract and expand for therapy force and/or storage. The rim 346 may be made from one part or more than one part. The rim 346 may be made from one material, more than one material or a combination of materials. The rim 346 may be configured to apply greater pressure in one area than in another area of the rim 346. For example, the rim 346 may be weighted, either by density, thickness, material, attachments, protrusions, etc., in one portion (e.g., one half of the cross-section of the device). This portion may be configured to apply a greater pressure than the remainder of the rim 346. The remainder of the rim 346 may be constructed similarly to the remainder of the device 300 (e.g., of the same material, density, thickness, etc.). The rim 346 may be tilted, lopsided, or angled to place pressure at different locations in the vaginal canal based on anatomy, type of incontinence, and/or prolapse. The upper end 305 may be open.

The upper portion 342 may include the rib 380. The upper portion 342 and/or the rib 380 may have a perimeter that is a circular transverse cross-section throughout its length. The circular transverse cross-section may reduce in diameter from the upper end 305 (D₁) to the junction 303 (D₂). The reduction in diameter may be a reduction in internal diameter of the upper portion 342, a reduction in the external diameter of the upper portion 342, or a combination thereof. The circumference of the upper portion 342 may decrease from the upper end 305 to the junction 303. The upper portion 342 may extend from the junction 303 to the upper end 305

The rib 380 may be configured to apply pressure through the wall 349 of the vaginal insert device 300 and onto an intravaginal or organ wall. The rib 380 may take on any shape configured to apply pressure to the intravaginal wall and/or support organs. In an embodiment, the rib 380 extends from adjacent the upper end 305 of the device 300 to the junction 303. In an embodiment, the rib 380 may be adjacent the rim 346. In an embodiment, the member or rib 380 may extend between two or more points on the interior side 350 of the wall 349. In an embodiment, the rib 380 extends from only one point on the interior side 350 of the wall 349. Any of the previously described or forthcoming descriptions, adjustments, and/or modifications to the rib 380 may be presented, alone or in combination, to the device 300 to apply pressure to the intravaginal wall and/or support organs in accordance with the principles of the current disclosure. The rib 380 may taper or decrease in diameter similar to the body 102 and/or the body 202. The rib 380 may be exposed between the wall 349 and the junction 303 such that no outer wall surrounds the rib 380 in this portion of the device 300.

The member or rib 380 is referred herein as a rib 380 for ease of disclosure. However, other structures or configurations are contemplated to achieve the application of pressure as the rib 380. The rib 380 may have four rib sections or members 380a, 380b, 380c, 380d. More or fewer rib sections or members are contemplated. The rib 380 may take on a variety of forms, some exemplary shapes, and arrangements of the rib 380 are described herein. Any combination of the exemplary shapes is contemplated.

The rib 380 may be cross-shaped, “T” shaped, or “X” shaped, although other shapes and arrangements are contemplated (such as, for example, a triangular shape, a pentagonal shape, a “Y,” “K,” “V” shape, etc.). The rib 380 may also be a star shape, meeting in one central location, in multiple locations or not meeting at all. The rib 380 may be one or more parallel ribs or members offset from the central axis, a single rib or member crossing the central axis, both the one or more parallel ribs or members offset from the central axis and the single rib or members crossing the central axis, and/or chords which extend from one interior surface to another interior surface without intersecting, etc., or any combination thereof. The rib 380 may be spokes or members which cross or meet at the center of the spokes or offset from the center of the spokes. The spokes or members may or may not meet the interior wall of the vaginal insert device. The rib 380 may be a member that may extend between diametrically opposing points on the interior wall of the upper portion 342. The member may connect to the interior wall at the diametrically opposing points. The rib 380 may include two or more members spaced equidistantly around a circumference of the interior wall of the cone-shaped body. The two or more members may meet at a center point of the cone-shaped body. The member may be a cross-shaped rib having four ends. Each of the four ends may meet the interior wall of the cone-shaped body. A device 300 of larger size may have more members or ribs than a device 300 of smaller size.

The vaginal insert device may collect vaginal discharge. The device may include internal ribbing features that may serve a purpose of sectioning-off vaginal fluids for diagnostic purposes. For example, if spotting is occurring, the vaginal insert may help a user determine the unseen source based on which cavity or section the blood or fluid is collected in. Indicator strips or nano-technology may be used parallel for diagnostic or treatment purposes. Medication may be used in one or all sections.

The rib 380 may be located adjacent the rim 346. The rib 380 may be horizontally aligned with the rim 346 and may be the same height as the rim 346 such that pressure is applied at the rim 346 (e.g., applied by both the rim 346 and the adjacent ribs 380). The rib 380 may take any shape or form that supports the vaginal insert device 300, applies force and/or pressure to organ walls, and/or prevents or inhibits prolapse of organs. The rib 380 may be any length, thickness, and/or width. The rib 380 may be offset from a central axis of the device 300. The rib 380 may allow for adjustable or selective pressure around a circumference of the device 300. For example, the location where the rib 380 approaches or meets with the upper portion 342 may apply a greater force to an organ wall than a location spaced apart from the rib 380. The device may be rotated to adjust pressure on the vaginal wall and/or organ walls. The device may allow for adjustment of the pressure applied, and thus the support provided, by the device.

Although described as a single rib 380, the rib 380 may be constructed of two rib portions or members that cross in the middle (e.g. a first rib portion or member of sections 380a, 380c crossing a second rib portion or member of sections 380b, 380d) or four rib portions or members that meet in the middle (e.g. rib sections 380a, 380b, 380c, 380d meeting in the middle). Where more than one rib member is provided, the rib members may be spaced equidistantly from one another. Alternatively, the rib members may be randomly located with respect to one another and/or may be offset such that one portion of the device 300 is configured to apply a greater pressure and/or greater support than the other side. That is, the side with more rib members may apply greater pressure or support than the side with fewer rib members. The rib 380 may be formed as an integral unit or as separate parts coupled together. The rib 380 may be made from one material, two or more materials or a combination of materials and/or parts. The rib 380 may be semi-permeable. The rib 380 may be formed integral with the upper portion 342 or may be formed as a separate component otherwise secured within the upper portion 342. The rib 380 may be removable from the upper portion 342. The entirety of the device 300, including the rib 380, may be formed as a single, unitary, integral device formed of a single material.

Although the rib 380 is shown with four members or rib sections 380a, 380b, 380c, 380d, more or fewer rib sections may be provided, e.g., one, two, three, four, five, size, seven, eight, nine, ten, or more rib sections are contemplated. The number of rib sections or members may be selected to adjust the amount of pressure applied to the organ wall, such as the uterine or bowel wall, to reduce, manage, improve, prevent, or eliminate incontinence. The rib sections or members (e.g., 380a, 380b, 380c, and 380d) may define one or more hollow or open portions 358. The device 300 includes four open portions 358.

The rib 380 may add support to the organs when pelvic organ prolapse occurs or to prevent organs from displacement. The rib 380 may provide additional pressure on the vaginal wall, urethral sphincter, and/or the bladder neck to assist in reducing, managing, improving, preventing, treating, and/or eliminating Incontinence. The rib 380 may also assist in preventing tissue from descending into the open portions 358 of the device. Over time, the tissue and/or organs may descend into the device 300. The inclusion of rib 380 may prevent this by blocking entry of the tissue into the device 300. The rib 380 may prevent sinking of the bladder, cervix, or rectum. The rib 380 may place sufficient pressure on the vaginal wall at the bladder neck or urethral sphincter through the intravaginal canal to effectively reduce leakage. The rim 346 and the rib 380 may, together, apply pressure and/or support, the organs and/or organ walls within the vagina.

The rib 380 may extend from adjacent or just below the rim 346 to the junction 303. In this manner, the rib 380 may apply pressure and support to the rim 346 and the upper portion 342, thus applying pressure and support to the uterine and/or rectum wall. The rib 380 may be surrounded by the wall 349 at an upper end of the upper portion 342.

Where rib 380 is provided, a softer or harder material may be used. The softer or harder material (e.g., silicone) may allow for more comfortable wearing while the rib 380 provides added pressure. That is, the rib 380 may accommodate for the pressure applied by a softer or harder material, thus allowing a more comfortable and effective device.

The lower portion 344 may be a stem, removal portion, and/or insertion portion. The lower portion 344 may assist in the insertion, removal, and/or positioning of the vaginal insert device 300. The lower portion 344 may be rod-shaped or cylindrical. The lower portion 344 may be hollow or solid. The lower portion 344 may have a constant diameter from the junction 303 to a lower end 304.

The lower portion 344 may include one or more ridges 348. The one or more ridges 348 may be spaced apart along an outer surface of the lower portion 344. The one or more ridges 348 may by spaced apart from the junction 303 of the upper portion 342 and lower portion 344. The one or more ridges 348 may be spaced uniformly and/or randomly spaced along the outer surface of the lower portion 344. The one or more ridges 348 may be semi-permeable to allow liquid, gas, lubrication, and/or medication to pass through. The ridges 348 may be circular rings that surround the exterior side of the lower portion 344. Alternatively, ridges 348 may include any protrusions that extend from the of the lower portion 344, such as studs, knobs, buttons, words, numbers, symbols, logos, other shapes, including polygonal, triangular, separated, randomly separated studs, uniformly spaced studs, or square, or combinations thereof. The ridges 348 may be helical, diagonal, longitudinal, or radially placed on the device 300. The ridges 348 may protrude at the same or different radially distances from the lower portion 344.

The upper portion 342 and the lower portion 344 may be fabricated as an integral, one-piece device formed of a single material. The upper end 305 may be integral with the upper portion 342 and the lower portion 344. For example, the upper portion 342 and/or the lower portion 344 may be formed of a medical grade silicone. The upper portion 342 and/or the lower portion 344 may be injection molded as a single, integral device of a medical grade silicone. Alternatively, the upper portion 342 and/or the lower portion 344 may be made from more than one part and/or more than one material that may attach and/or detach or expand and retract for therapy, treatment, or sterilization purposes. The upper portion 342 and the lower portion 344 may be permanently or detachably coupled together.

The vaginal insert device 300 may come in different sizes, density, shapes, durability and/or different durometers to accommodate women with differing anatomy, women with changing or fluctuating anatomy, to accommodate different uses of the device, and/or different activities performed while using the device. Furthermore, the dimensions of the various sections and portions of the device 300 may be modified from the multiple embodiments illustrated and disclosed herein. For example, the total height of device 300, diameter at the upper end of the upper portion 342, thickness of the wall of the upper portion 342 of the device 300, diameter of the lower end 304, and/or height of the lower portion 344 may be modified. Modifications made to the dimensions may still retain or may improve on the intended usefulness, effectiveness, and other benefits of the device. The dimensions may be modified in any combination or individually.

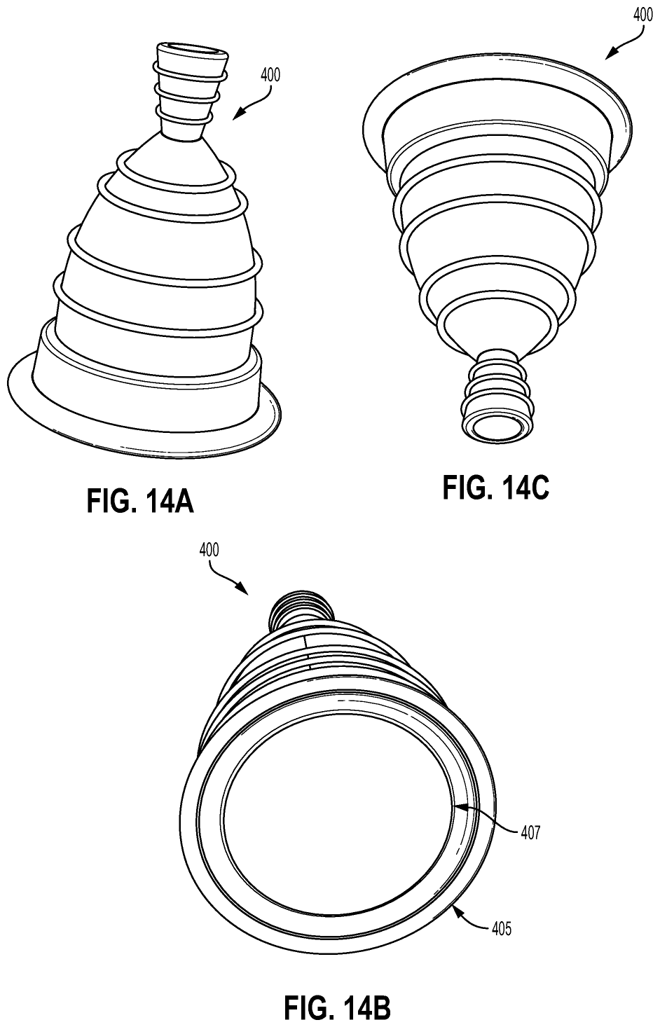

Referring now to FIGS. 14A to 14C, an exemplary vaginal insert device 400 is shown. The vaginal insert device 400 is similar to the device 200. The vaginal insert device 400 may include any of the modifications, shapes, sizes, materials, dimensions, uses, or any combinations thereof as described with respect to the vaginal insert device 100 and/or device 200. Any of the modifications or alternatives of the rim, stem, shape of the upper portion, etc., previously described may be applied separately or in combination to the vaginal insert device 400. The method of insertion and use as described in FIGS. 6-10 may apply to the vaginal insert device 400. The vaginal insert device 400 may have all the same components and features of the vaginal insert device 200. In the vaginal insert device 400, the surface 407 may be solid with no opening such that the upper end 405 is completely closed and no fluid, tissue, air flow, or any other material or fluid is permitted to enter the upper end 405.

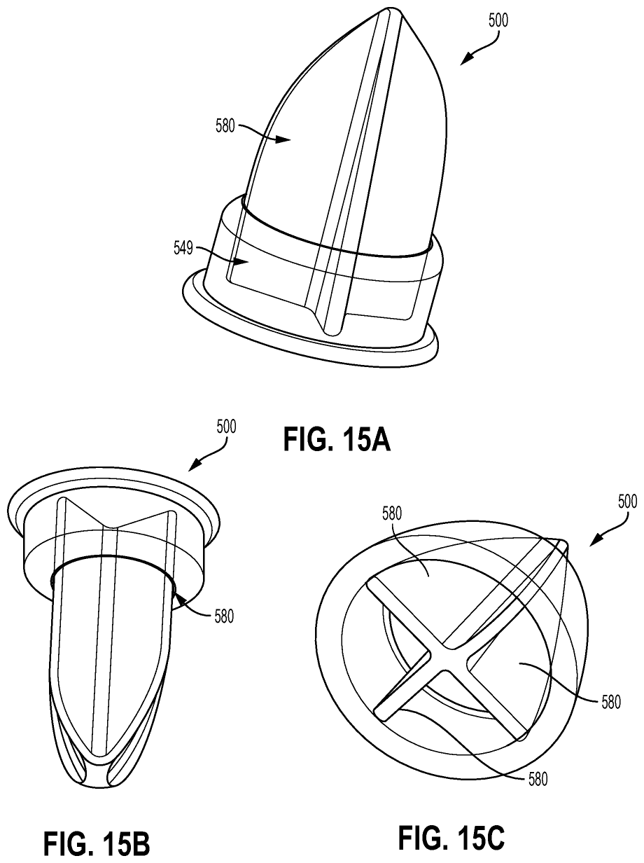

Referring now to FIGS. 15A to 15C, an exemplary vaginal insert device 500 is shown. The vaginal insert device 500 is similar to the device 300. The vaginal insert device 500 may include any of the modifications, shapes, sizes, materials, dimensions, uses, or any combinations thereof as described with respect to the vaginal insert devices 100, 200, 300, and/or 400. Any of the modifications or alternatives of the rim, stem, shape of the upper portion, etc., previously described may be applied separately or in combination to the vaginal insert device 500. The method of insertion and use as described in FIGS. 6-10 may apply to the vaginal insert device 500. The vaginal insert device 500 may have all the same components and features of the vaginal insert device 300. In the vaginal insert device 500, the lower portion (e.g. 344 of FIG. 13A) may be omitted. Although the vaginal insert device 500 is shown as two components (e.g. wall 549 and rib 580) coupled together, the device 500 may be formed as a single, integral component. The variations of the rib 380 previously discussed may be provided to the rib 580.

The vaginal insert device of the present disclosure may provide additional or alternative functions to pressure application and/or organ support. For example, the vaginal insert device of the present disclosure may be used to administer medication or topical treatments to the vaginal tissue. Various configurations are contemplated herein. A medical device for administering medication may be used alone or in combination with the features that address urinary incontinence, POP, or both. Various medications and topical treatments, that are described in more detail below, may be used with the vaginal insert device. A medical device for administering medication may be used alone or in combination with the features that collect vaginal fluids, discharge and/or tissue samples, as well as the features for detection and/or diagnosis of various diseases and/or medical conditions. Various medications and topical treatments, that are described in more detail below, may be used with the vaginal insert device.

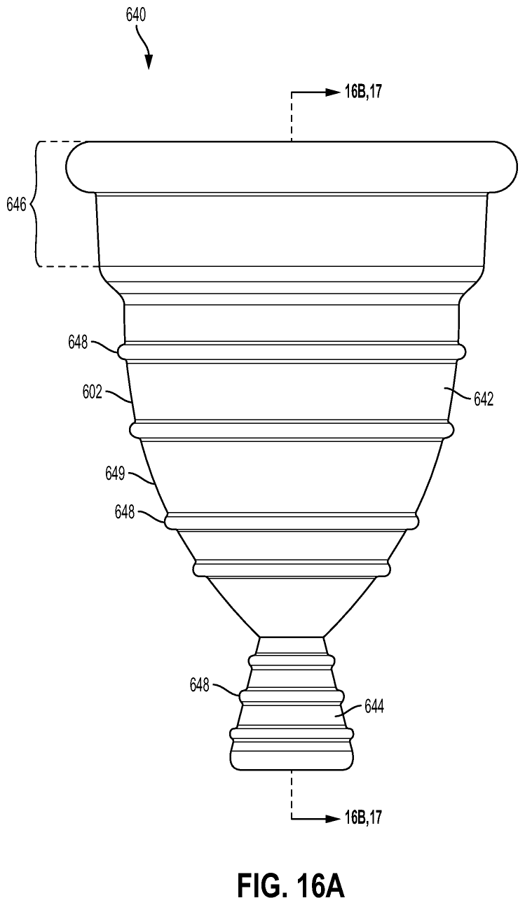

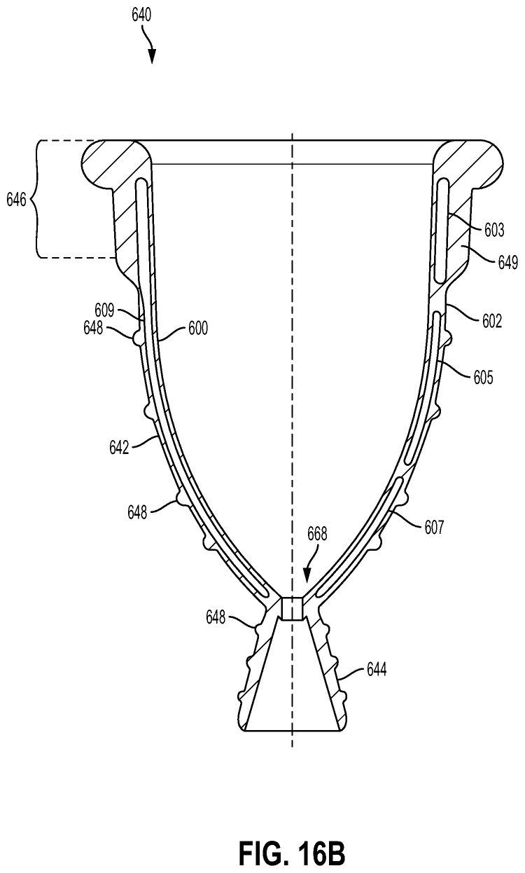

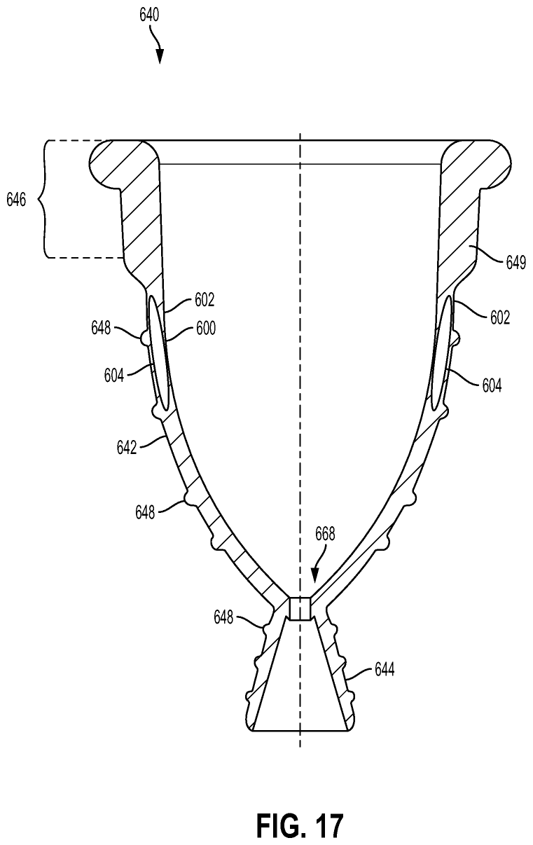

An exemplary vaginal insert device 640 for use with medication is shown in FIGS. 16A and 16B and FIG. 17. As shown in FIGS. 16A and 16B, the vaginal insert device 640 may have a wall 649 with an inner surface 600 and an outer surface 602. The wall 649 may be hollow or semi-hollow. In some examples, a top portion of the upper portion 642, a middle portion of the upper portion 642, and/or bottom portion of the upper portion 642 of the wall 649 may contain a cavity (e.g., cavity 603 in the top portion, cavity 605 in the middle portion, and/or cavity 607 in the lower portion). In some examples, a cavity 609 may run throughout wall 649. Although FIG. 16B shows different cavities on different sides, it may be appreciated that the same cavity may present throughout the entirety of the wall 649. The cavity may extend circumferentially and/or annularly within the wall 649 around all or a portion of the circumference of the device 640. Any combination of the cavity may be present in all or in partial-circumferential portions of the wall 649. The portion(s) of wall 649 containing a cavity may have a perforated outer surface 602. The outer surface 602 may be the surface of wall 649 that faces the vaginal tissue when the device 640 inserted.

The inner surface 600 may have a permeability that is different from the outer surface 602. That is, the inner surface 600 may be more permeable or less permeable than the outer surface 602. Some or all of the inner surface 600 may have a different permeability than the some or all of the outer surface 602. For example, all, or portions of (e.g., at the cavity or at portions not at the cavity), the inner surface 600 may be less permeable than all, or portions of, the outer surface 602. For example, the portion of wall 649 containing a cavity (e.g., cavity 603 in the top portion, cavity 605 in the middle portion, and/or cavity 607 in the lower portion) may hay have a non-permeable or less permeable inner surface 600. For example, inner surface 600 of the portions of the wall 649 containing the cavity may not have perforations. Outer surface 602 of the portions of the wall 649 containing the cavity may have perforations. In some examples, outer surface 602 of wall 649 may be comprised of a more permeable material than the inner surface 600. This may allow for a substance to be transmitted through outer surface 602 more readily than inner surface 600.

In some examples, all, or portions of, the inner surface 600 may be more permeable than all, or portions of, the outer surface 602. For example, the inner surface may be more permeable by including perforations, being formed of a more permeable material than the outer surface 602, etc., or combinations thereof. In examples having the inner surface 600 is more permeable than the outer surface 602, the vaginal insert device 640 may collect samples (e.g., vaginal fluids and/or tissues) within the interior of the vaginal insert device 640. The samples may be analyzed, tested, or diagnosed in accordance with the present disclosure. In examples having the inner surface 600 more permeable at the location of a cavity, the cavity may collect the samples within the cavity.

Thus, the cavity may function as a reservoir for a medicine or treatment and/or for collection of a vaginal fluids and/or tissues. The vaginal insert device 640 may include multiple cavities with the walls of each cavity having a different permeability such that one or more cavities function to deliver a medicine or treatment (e.g., outer surface 602 is more permeable at the cavity than the inner surface 600) and one or more cavities function to collect vaginal fluid and/or tissue (e.g., inner surface 600 is more permeable at the cavity than the outer surface 602). In this manner, the vaginal insert device 640 may operate as both a delivery and collection device.

In some examples, the cavity may contain reagents that react when vaginal fluid and/or tissue is absorbed (or permitted to flow through, e.g., via perforations) through the inner surface 600. The reagents may react with the vaginal fluid and/or tissue. The reaction may provide an indication of a medical condition and/or health condition (e.g., bacterial infection, ovulation, sexually transmitted disease, vitamin level of the user, vitamin deficiency, other conditions described herein, etc.). The indication may be a visual indication. In some examples, the inner surface 600 may change color to provide the indication (e.g., to reflect the results of the diagnostic test). In some examples, the vaginal insert device 640 may include a microchip, Bluetooth, wireless transmission device, or other communication or transmission device to electronically transmit the health outcome to the user.

In some embodiments, the inner surface 600 may be removable from the outer surface 602. In further embodiments, only a portion of the outer surface 602 may be removable from inner surface 600. In either embodiment, the cavity may be filled with a substance further described herein. A substance may be injected or permeate into the cavity through inner surface 600 or outer surface 602. The substance may be a liquid, solid, powder, gel, or some combination thereof.

The substance may be absorbed or emitted from the cavity through the outer surface 602 into the vaginal tissue when inserted. The substance may be topical to minimize discomfort caused by the presence of the vaginal insert device 640 or may be intended to treat another condition. In some embodiments, the substance may have a medicinal property that is realized when the substance is absorbed through, or applied to, vaginal tissue. In embodiments, the substance may include an anti-bacterial, anti-microbial, or anti-inflammatory property. The substances may further include a combination of a number of substances described herein.

The cavity may run throughout wall 649, such as, for example, cavity 609. In embodiments where the cavity runs throughout wall 649, the cone shape of the device may secrete the medication throughout the entire length of the vaginal canal rather than just in one spot. In other embodiments, such as shown in FIG. 17, a partial cavity 604 may be limited to portions of wall 649, such as the portions described with respect to the right-hand side of FIG. 16B and cavity 603, cavity 605, cavity 607. The partial cavity 604 may extend along any longitudinal portion of the wall 649. For example, in FIG. 17, partial cavity 604 is in the middle portion of the insert. In other embodiments, the substance may be in a cavity within rim 646, ridges 648, in the upper portion 642, in the lower portion 644, in the of vaginal insert device 640. In other embodiments, the substance may be in one or more cavities that run along the length of vaginal insert device 640. If the cavity runs throughout wall 649, inner surface 600 and outer surface 602 may be perforated or permeable along portions or the entirety of wall 649. If the cavity runs through only portions of wall 649, such as partial cavity 604, the inner surface 600 may be less permeable than the portions of wall 649 that do not contain the cavity. Further, the outer surface 602 may be more permeable than portions of wall 649 that do not contain the cavity. In this way, the portions of wall 649 that do not contain a cavity may allow for ventilation through inner surface 600 and outer surface 602. Further, the portions of the wall 649 that contain the cavity may allow emission of a substance in the cavity through outer surface 602 and may limit the emission of a substance into the interior of vaginal insert device 640 through inner surface 600.

The cavity may be in any shape or size within wall 649. For example, the cavity may not extend along the circumference of wall 649. In other embodiments, the cavity in the wall may extend 360 degrees around the device. Further, there may be multiple cavities within wall 649. The cavities may be arranged to optimize the absorption of the substance while allowing for ventilation through the portions of wall 649 that do not contain the cavity. The perforations, permeable material, size, shape, or placement may vary depending upon the intended use. For example, the permeable material, perforation size, or cavity shape may depend on the recommended dosage or chemical characteristics of the substance. In some embodiments, a ring having a permeable membrane may be inserted into the wall 649 or may be placed around the exterior circumference of vaginal insert device 640. A ventilation opening 668 may or may not be included. The vaginal insert device 640 may have any or all of the structure or features of any of the vaginal insert devices described herein.

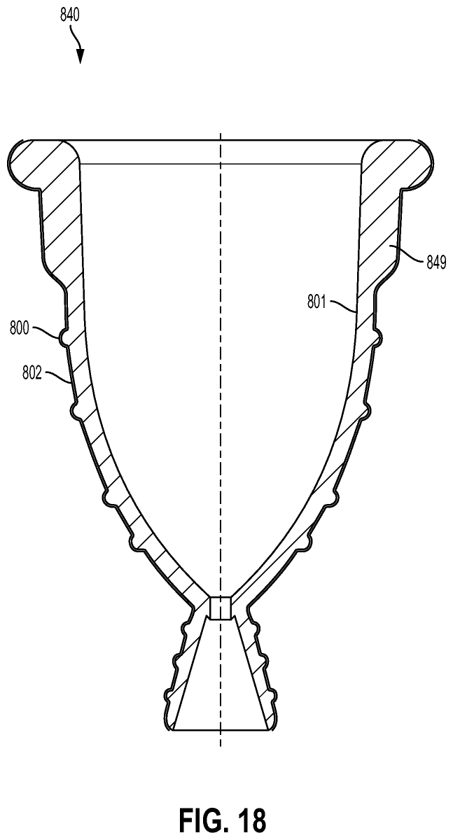

In another aspect of the disclosure a vaginal insert device 840 may include medication(s), composition(s), reagent(s), formulation(s), and/or substance(s) on a surface of the device as shown in FIG. 18. For example, the device may be dipped and/or coated in a substance 800. The substance 800 may include, but is not limited to, a medication, a reagent, a treatment, a vitamin, other type of composition having properties, or any combination thereof. The substance 800 may be included to improve, address, treat, monitor, analyze, or combinations thereof, symptoms, health, and/or medical conditions of the user. For example, the vaginal insert device 840 may include a treatment or vitamin to be delivered to the user. In some examples, the vaginal insert device 840 may include a reagent to react with vaginal fluid and/or vaginal tissue that is absorbed, permitted to flow through, or comes in contact with the vaginal insert device. This may produce a visual indicator of a health outcome, health status, medical condition, status of the device (e.g., the need to remove the device, the need to change the device to a new device, etc.) etc. In some examples, the device may or may not be hollow or contain a cavity. Further, in these embodiments the device walls may or may not be permeable. The device may have a wall 849 with smooth inner surface 801 and/or may have a smooth or a ribbed outer surface 802. A smooth surface may be a surface with no surface features. The vaginal insert device 840 may have the structure of any of the vaginal insert devices described herein.

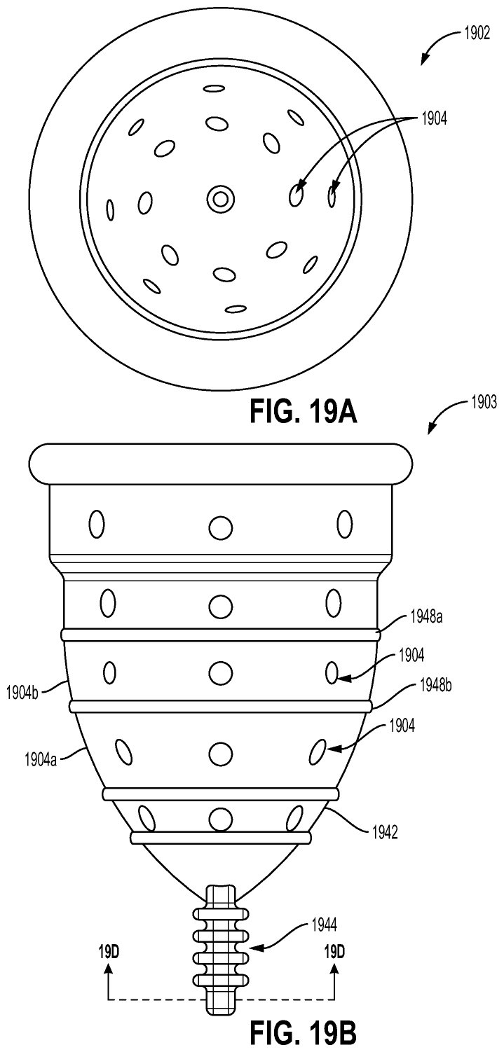

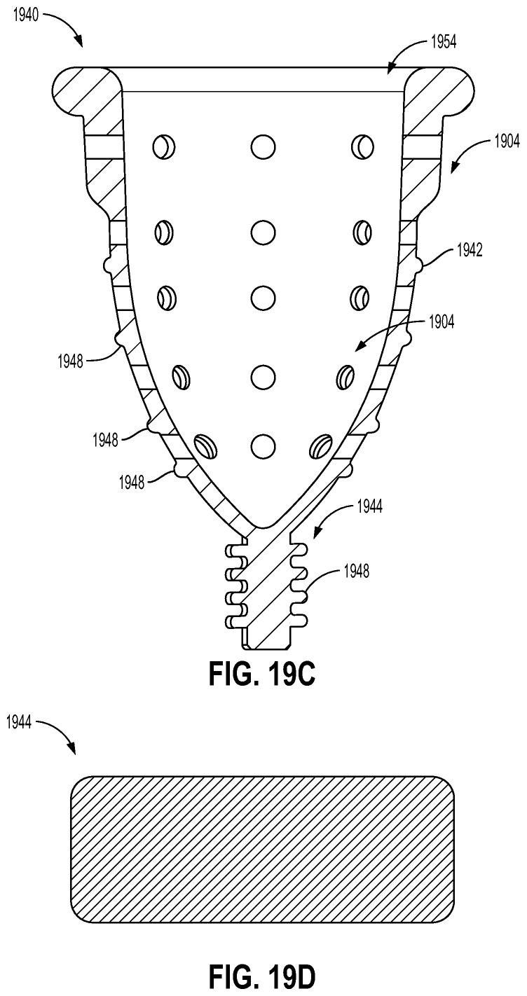

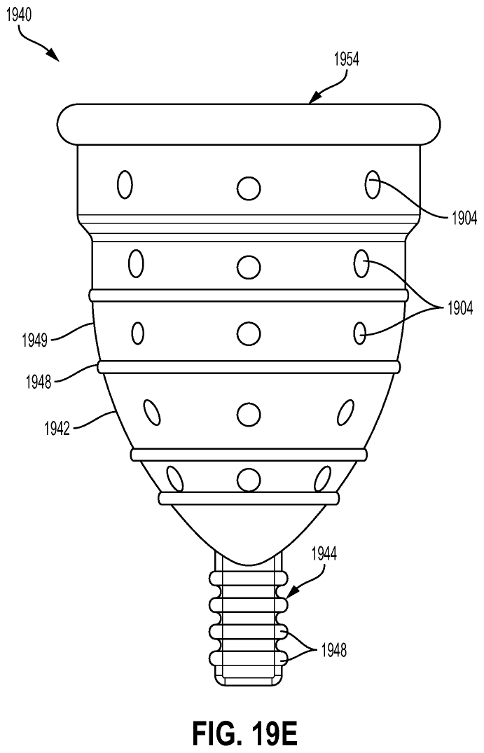

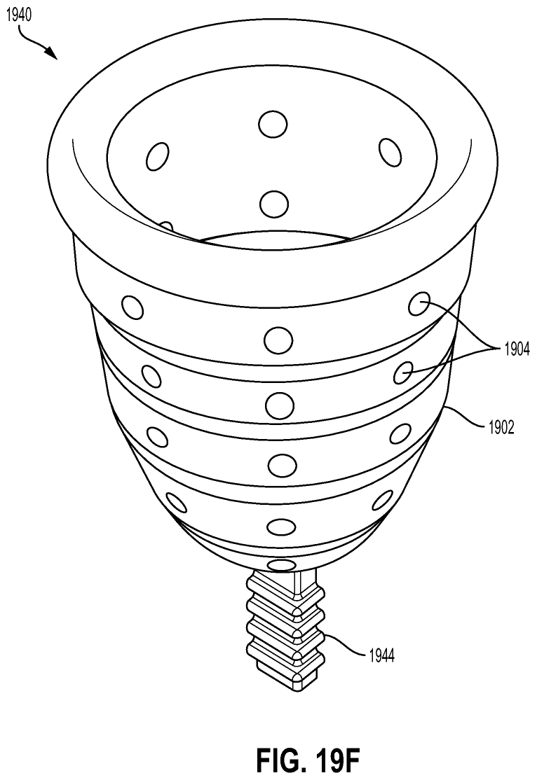

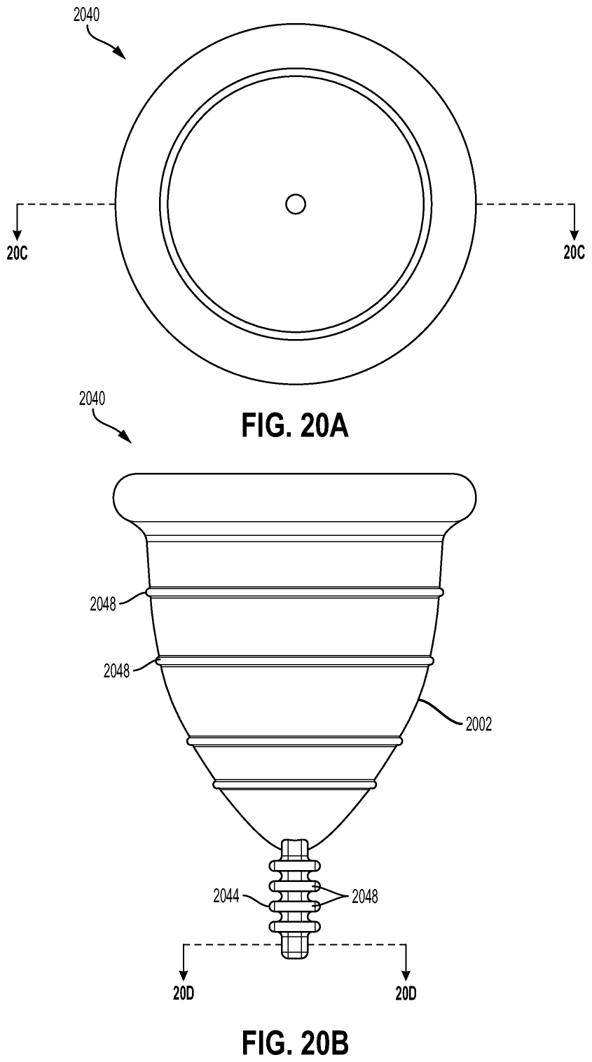

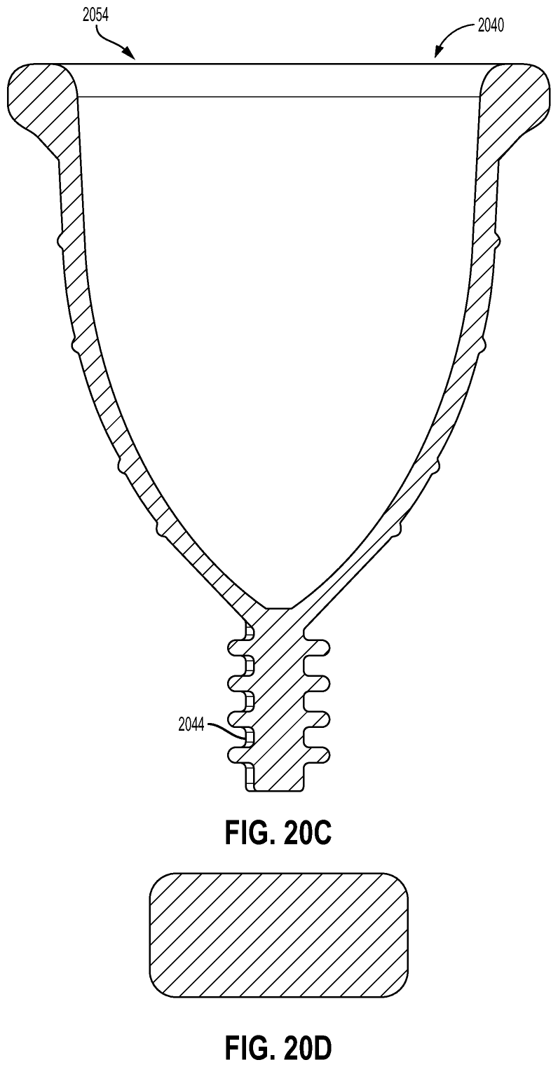

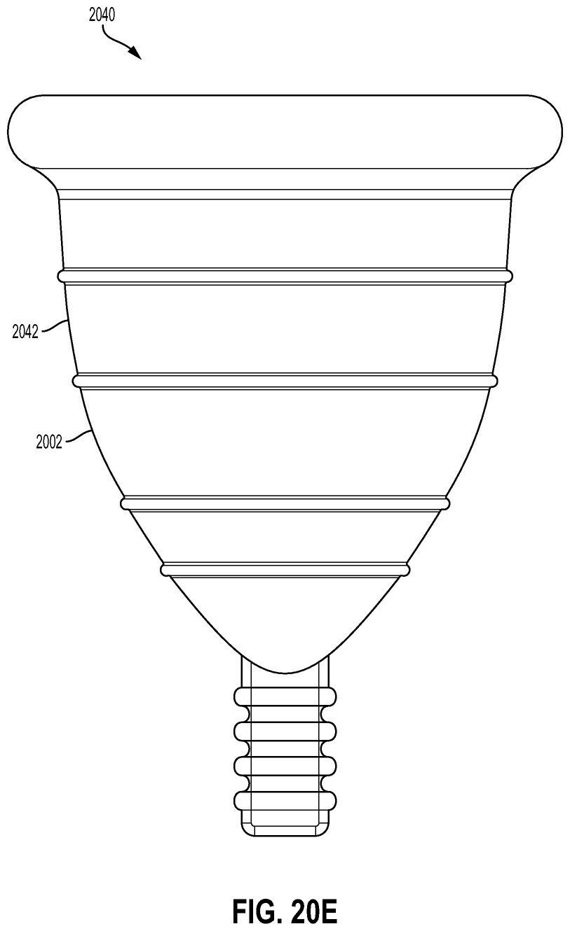

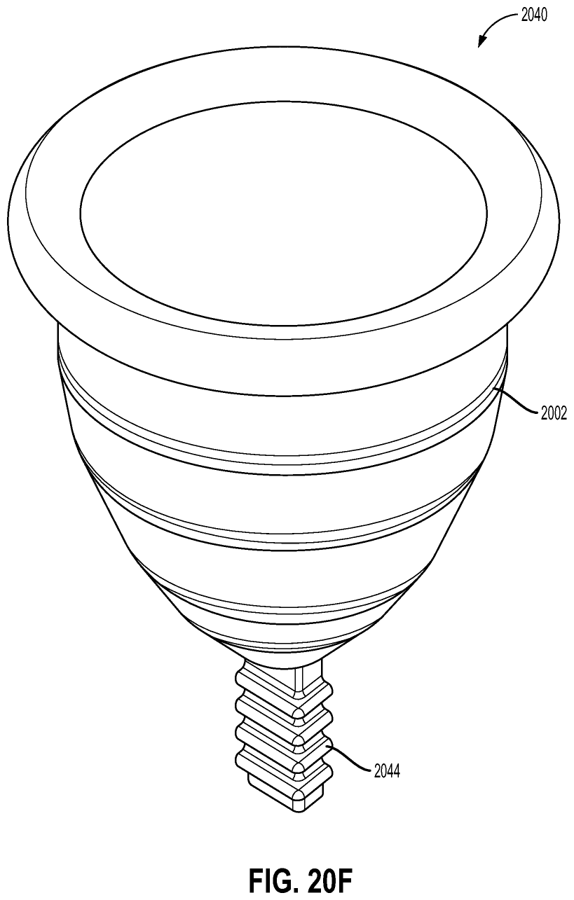

FIGS. 19A to 19F and FIGS. 20A to 20F show vaginal insert devices in accordance the principles in the present disclosure. In one aspect of the disclosure, these devices preferably may be used to administer medication and/or topical treatments to the vaginal tissue, such as described herein. This functionality may be provided alone and/or in combination with the other functionalities described in this disclosure. Various measurements are shown and described herein and in the figures, but are not intended to be limiting. Various measurements are contemplated for use with the device.

FIGS. 19A to 19F depict a vaginal insert device 1940 that may be configured to accommodate medication(s), composition(s), reagent(s), formulation(s), and/or substance(s), as described herein. For example, the vaginal insert device 1940 may accommodate a medicinal substance or ointment, topical or medicinal substance, or may be configured to be filled with a medicinal ointment or substance. As shown, FIGS. 19A to 19F, the vaginal insert device 1940 may include perforations 1904, also referred to as openings 1904, in a wall 1949 of the vaginal insert device 1940. The medication, composition, reagent, formulation, substance, medicinal substance or ointment, topical or medical substance, etc. may be deposited and/or housed, in part, or in whole within one or more of the perforations 1904. In this manner, the substance disposed within the perforations 1904 may be exposed to either the interior of the vaginal insert device 1940 and/or the exterior of the vaginal insert device 1940 (e.g., the interior of the vagina). This may allow for delivery of the substance, collection of a substance, and/or diagnostic with the substance (e.g., reaction of a fluid/tissue with the reagent to provide a diagnosis and/or indication of a condition to the user). The vaginal insert device 1940 may have any or all of the features of any of the vaginal insert devices described herein, with the addition of perforations 1904.

The vaginal insert device 1940 may include a cone-shaped body 1902, as has been described herein. A lower portion 1944 may be connected at a lower end of the body 1902. An upper portion 1942 of the vaginal insert device 1940 may have an open upper end 1954. Alternatively, the upper end may be closed. The upper portion 1942 and/or the lower portion 1944 may include a plurality of ridges 1948. The plurality of ridges 1948 may be arranged in rows. The perforations 1904 may be arranged in rows circumferentially around the upper portion 1942. For example, rows 1904a and 1904b are shown in FIG. 19B. The rows of the perforations 1904 may be located between two adjacent ridges 1948. For example, row 1904b of the perforations 1904 may be located between ridge 1948a and ridge 1948b. The lower portion 1944 may be a flat body. The lower portion 1944 may extend downward from a lower surface of the upper portion 1942. The lower portion 1944 and the ridges 1948 thereon may facilitate insertion and removal of the vaginal insert device 1940.

Referring to FIG. 19F, a perspective view of a perforated embodiment of the vaginal insert device 1940 is shown. FIG. 19A is a top view of the vaginal insert device 1940. An outer diameter of the top of the upper portion 1942 may be between 37.75 mm and 38.25 mm. A height 1903 of the vaginal insert device 1940 may be between 57.15 mm and 57.65 mm. The perforations 1904 may extend through the wall 1949 (e.g. from the outer surface through the inner surface) of the vaginal insert device 1940. In some examples, the perforations 1904 may extend only partially through the wall 1949 (e.g., through the outer surface, through the inner surface, or combinations thereof) through the vaginal insert device 1940. For example, the outer surface may be more perforated than the inner surface or vice-versa. FIG. 19C is a cross section of the vaginal insert device 1940 taken along line A-A of FIG. 19A. As shown in FIG. 19C, the perforations 1904 may pass through the wall of the vaginal insert device 1940 so that the perforations are open to the interior and exterior of the vaginal insert device 1940. In other embodiments, the perforations 1904 may be sealed on the interior so that they are only open at the exterior. In this way any medication may be inhibited from leaking into the interior of the device and may only exit through the exterior surface of the device to the intravaginal tissue. In still further embodiments, there may be a seal on the inner- and outer surface of the device and the seal on the outer surface of the device may be dissolvable using any of the seals disclosed herein or otherwise know in the art. The arrangement, quantity and/or sizes of perforations 1904 may vary from that shown. Depending upon the medication and/or targeted areas for delivery of the medication, the arrangement, quantity and/or sizes of the perforations may be configured and/or arranged to target specific areas of the tissue, for example.

FIGS. 19B and 19E are a front view and side view of the vaginal insert device, respectively. Lower portion 1944 may facilitate removal of the vaginal insert device when downward pressure is applied to the lower portion 1944. Lower portion 1944 may be ribbed or have ridges that facilitate gripping the vaginal insert device for removal, such as described herein. The ridges may be spaced between 2.25 mm and 2.75 mm apart. FIG. 19D is a cross-sectional view of the lower portion 1944 taken along line 19D-19D of FIG. 19B. The lower portion 1944 may be between 10.75 mm and 11.25 mm long, between 6.75 mm and 7.25 mm wide, and between 4.25 mm and 4.75 mm thick. The above are exemplary dimensions, however other dimensions are contemplated herein, as described and shown in the various other configurations described and shown herein.

FIGS. 20A to 20F depict another embodiment of a vaginal insert device 2040 that does not have perforations. The vaginal insert device 2040 may be the same as the vaginal insert device 1940, without the inclusion of perforations 1904 (FIG. 19A). The vaginal insert device 2040 may be used to administer medication and/or topical treatments to the vaginal tissue. This functionality may be provided alone and/or in combination with the other functionalities describe in this disclosure. Medications may be included and/or mixed in the material itself of the device, applied to the surface of the device, such as by being coated or dipped with medication, disposed in a cavity or pocket in the device, such as, in the wall, rim or tip of the device, and/or disposed in a cavity or pocket in the device where the medicine may pass from the cavity, through pores in the wall of the device into contact with the tissue.

The vaginal insert device 2040 may include a cone-shaped body 2002, as has been described herein. A lower portion 2044 may be connected at a lower end of the body 2002. An upper portion 2042 of the vaginal insert device 2040 may have an open upper end 2054. Alternatively, the upper end may be closed. The upper portion 2042 and/or the lower portion 2044 may include a plurality of ridges 2048. The plurality of ridges 2048 may be arranged in rows. The lower portion 2044 may be a flat body. The lower portion 2044 may extend downward from a lower surface of the upper portion 2042. The lower portion 2044 and the ridges 2048 thereon may facilitate insertion and removal of the vaginal insert device 2040.

Any of the body 2002, lower portion 2044, upper portion 2042, ridges 2048, or any combination thereof may be impregnated, coated, covered, or otherwise supplied with a medicine or substance as described herein.

In the configuration shown in FIGS. 20A to 20F, the rim of the upper portion may be between 43.75 mm and 44.25 mm in diameter. The vaginal insert device 2040 may be between 58.52 mm and 59.02 mm long. The lower portion may be between 12.40 mm and 12.90 mm long, between 6.40 mm and 6.80 mm wide, and between 4.93 mm and 5.43 mm thick. The lower portion may also include ridges spaced between 2.63 mm and 3.13 mm apart.

Although not shown in FIGS. 19A to 19F and FIGS. 20A to 20F, a space or cavity may exist between the inner surface and the outer surface, such as described with respect to FIGS. 16A, 16B and 17. The size, length and/or width of the space or cavity may vary. The space may include a medicinal substance or ointment, topical or medicinal substance, or it may be configured to be filled with a medicinal ointment or substance. The medicinal ointment or substance may be absorbed or osmosed by the intravaginal wall through the wall of the device, including, for example, through perforations in the wall. In other embodiments, the pressure from the intravaginal wall on the vaginal insert device may cause the substance to be excreted over time.

The dimensions depicted in FIGS. 19A to 19F and 20A to 20F are for exemplary purposes only. Other dimensions are contemplated, and these dimensions do not limit the breadth of the application.

The device shown and described in FIGS. 19A to 19F and 20A to 20F, as well as the other devices described herein, may be coated in medication by dipping or spraying. The device may be configured and/or manufactured to heat or cool for pain relief, for example. The device may include medication in an internal hollow portion. The medication may include time release medication that is in the silicone which is done during the liquid injection molding process. The device may include one or more types of medications. The one or more medications may include medications located in the perforations, on the surface, in the cavity, inside the cone, and/or a combination of those locations of the device.

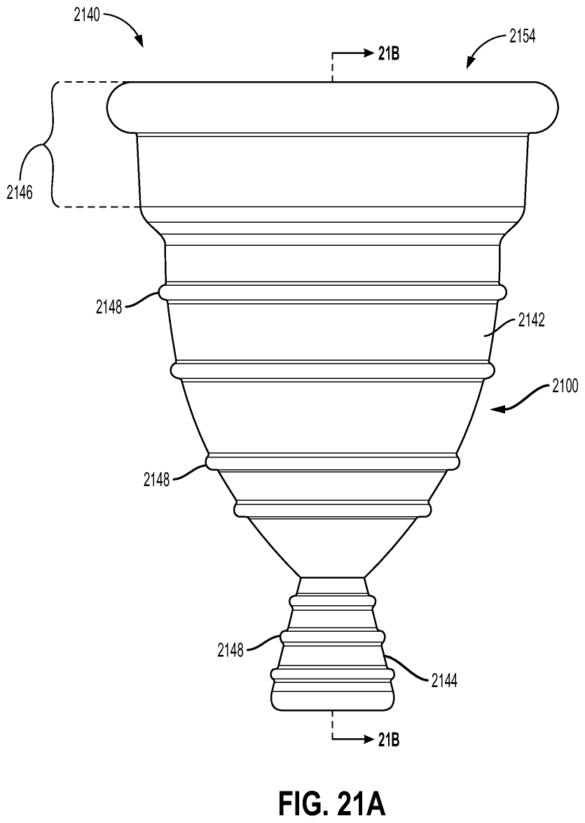

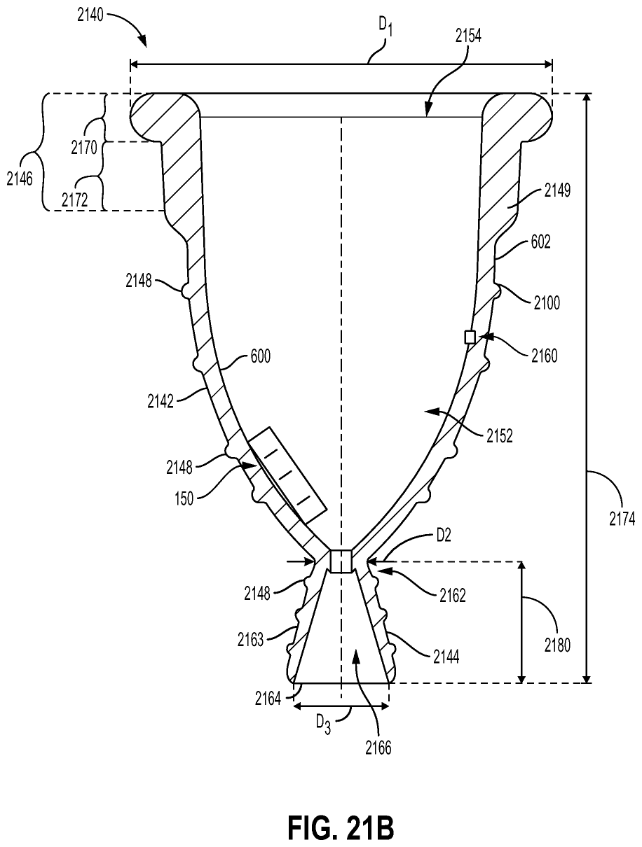

Referring to FIGS. 21A and 21B, further details of an exemplary vaginal insert device 2140 are shown. The vaginal insert device 2140 may include a body 2100. The body 2100 includes the upper portion 2142 and the lower portion 2144. The upper portion 2142 may be cone-shaped and/or cup-shaped. The upper portion 2142 is generally hollow within the walls of the upper portion 2142 (see, e.g., FIG. 21B in which inner surface 600 of the upper portion 2142 provide a hollow interior 2152) in order to collect a sample of vaginal fluid and/or discharge. The upper portion 2142 may have a first diameter D₁ which decreases to a second diameter D₂. The decreasing in diameter may be gradual. The upper portion 2142 may include a changing diameter. Although described herein as cone-shaped, other shapes of the upper portion 2142 are contemplated. Such other shapes may include changing diameters such that a first diameter is configured to apply pressure to an intravaginal or organ wall and a second diameter is configured to be smaller than the first diameter and not interact with an intravaginal wall. Such shapes may include trapezoid, spheres, cylinders, cubes, pyramids, hexagonal prisms, etc.

The upper portion 2142 may include a rim 2146. The rim 2146 may protrude from the upper portion 2142. The rim 2146 may be circular and may surround and protrude from the exterior side of the body 2100. The rim 2146 may be adjacent to an open, upper end 2154. The rim 2146 may be adjustable in height, location, and/or width. The rim 2146 may retract and expand for therapy force and/or storage. The rim 2146 may be made from one part or more than one part. The rim 2146 may be made from one material, more than one material or a combination of materials. The rim 2146 may be configured to apply greater pressure in one area than in another area of the rim 2146. For example, the rim 2146 may be weighted, either by density, thickness, material, attachments, protrusions, etc., in one portion (e.g., one half of the cross-section of the device). This portion (see, e.g., portion 2170 of FIG. 21B) may be configured to apply a greater pressure than the remainder of the rim 2146 (see, e.g., remainder portion 2172 of FIG. 21B). The remainder of the rim 2146 may be constructed similarly to the remainder of the device 2140 (e.g., of the same material, density, thickness, etc.). The rim 2146 may be tilted, lopsided, or angled to place pressure at different locations in the vaginal canal based on anatomy, type of incontinence, and/or prolapse.

The upper portion 2142 may include one or more ridges 2148. The one or more ridges 2148 may be spaced apart along an outer surface of the upper portion 2142. The one or more ridges 2148 may by spaced apart from a junction 2162 of the upper portion 2142 and lower portion 2144 to the rim 2146. The one or more ridges 2148 may be spaced uniformly and/or randomly spaced along the outer surface of the upper portion 2142. The one or more ridges 2148 may be semi-permeable to allow liquid, gas, lubrication, and/or medication to pass through. The ridges 2148 may be circular rings that surround the exterior side of the body 2100. Alternatively, ridges 2148 may include any protrusions that extend from the of the body 2100, such as studs, knobs, buttons, words, numbers, symbols, logos, other shapes, including polygonal, triangular, separated, randomly separated studs, uniformly spaced studs, or square, or combinations thereof. The ridges 2148 may be helical, diagonal, longitudinal, or radially placed on the device 2140 (either the upper portion 2142 and/or the lower portion 2144). The ridges 2148 may be the same or different on the upper portion 2142 and the lower portion 2144.

The upper portion 2142 may extend from the junction 2162 to an upper end 2154. According to one embodiment, the upper end 2154 is open in order to collect a vaginal fluid and/or discharge sample. The upper end 2154 may be formed of a material that is the same or similar as the body 2100. According to another embodiment, the upper end 2154 could be closed (not shown) in order to collect a discharge or tissue sample along an exterior of the body 2100 of the device 2140.

The upper portion 2142 may have a circular transverse cross-section throughout its length. The circular transverse cross-section may reduce in diameter from the upper end 2154 (D₁) to the junction 2162 (D₂). The reduction in diameter may be a reduction in internal diameter of the upper portion 2142, a reduction in the external diameter of the upper portion 2142, or a combination thereof. The circumference of the upper portion 2142 may decrease from the upper end 2154 to the junction 2162.

The lower portion 2144 may be a stem, removal portion, and/or insertion portion. The lower portion 2144 may assist in the insertion, removal, and/or positioning of the vaginal insert device 2140. The lower portion 2144 may be cone-shaped or cup-shaped. The lower portion 2144 may increase in diameter from D₂ to a second diameter D₃. The lower portion 2144 may include a lower end 2164. The lower end 2164 may be closed in order to collect the vaginal fluid and/or discharge sample within the device 2140. That is, there is a surface and/or wall closing the end 2164. The lower portion 2144 may be hollow or solid within the walls of the lower portion 2144.

The lower portion 2144 may include one or more ridges 2148. The one or more ridges 2148 may be spaced apart along an outer surface of the lower portion 2144. The one or more ridges 2148 may by spaced apart from the junction 2162 of the upper portion 2142 and lower portion 2144 to the lower end 2164 of the lower portion 2144. The one or more ridges 2148 may be spaced uniformly and/or randomly spaced along the outer surface of the lower portion 2144. The one or more ridges 2148 may be semi-permeable to allow liquid, gas, lubrication, and/or medication to pass through. The one or more ridges 2148 may be the same or similar to the one or more ridges 2148 on the upper portion 2142.

The upper portion 2142 and the lower portion 2144 may be fabricated as an integral, one-piece device formed of a single material. The lower end 2164 may be integral with the upper portion 2142 and the lower portion 2144. For example, the upper portion 2142, the lower portion 2144, and/or the lower end 2164 may be formed of a medical grade silicone. The upper portion 2142, the lower portion 2144, and/or the lower end 2164 may be injection molded as a single, integral device of a medical grade silicone. Alternatively, the upper portion 2142, the lower portion 2144, and/or the lower end 2164 may be made from more than one part and/or more than one material that may attach and/or detach or expand and retract for therapy, treatment, or sterilization purposes. The upper portion 2142 and the lower portion 2144 may be permanently or detachably coupled together.

The vaginal insert device 2140 may come in different sizes, density, shapes, durability and/or different durometers to accommodate women with differing anatomy, women with changing or fluctuating anatomy, to accommodate different uses of the device, and/or different activities performed while using the device. Furthermore, the dimensions of the various sections and portions of the device 2140 may be modified from the multiple embodiments illustrated and disclosed herein. For example, the total height of device 2140 (e.g., height 2174 of FIG. 21B), diameter at the upper end 2154 of the upper portion 2142 (e.g., diameter D₁ of FIG. 21B), diameter at the junction 2162 between the upper portion 2142 and the lower portion 2144 (e.g., diameter D₂ of FIG. 21B), thickness of the wall 2149 of the upper portion 2142 of the device 2140 (e.g., thickness between the outer surface 602 and inner surface 600 of the wall 2149), thickness of the wall 2160 of the lower portion 44 of the device 2140, diameter of the lower end 2164 (e.g., diameter D₃ of FIG. 21B), and/or height of the lower portion 2144 (e.g., height 2180 of FIG. 21B), may be modified. Modifications made to the dimensions may still retain or may improve on the intended usefulness, effectiveness, and other benefits of the device. The dimensions may be modified in any combination or individually.

According to one embodiment, the vaginal insert device 2140 may collect vaginal fluid, discharge, blood, etc. within the hollow interior 2152 of the upper portion 2142. The vaginal insert device may also collect vaginal fluid, discharge, blood, etc. within a hollow interior 2166 of the lower portion 2144. The device 2140 may include internal ribbing features that may serve a purpose of sectioning-off vaginal fluids for diagnostic purposes. For example, if spotting is occurring, the vaginal insert may help a user determine the unseen source based on which cavity or section the blood or fluid is collected in.

In still further embodiments, the vaginal insert device 2140 can be used to collect vaginal fluid, blood, discharge, etc., and the collected fluids may thereafter be used for detection and/or diagnosis purposes. The vaginal insert device 2140 may be further equipped with a sensor that may detect properties, conditions, or health of the vaginal fluid and/or tissue. The sensor may be a reagent, an indicator strip, an electrical sensor, a receptor, etc. For example, the sensor may detect properties, such as pH value, the presence of a bacteria or microbe, vitamin level, ovulation, etc. The property may be communicated to the user. In some examples, the property and/or diagnosis is provided to an external monitoring device, such as, e.g., an application or a medical provider system. In some examples, the device 2140 provides a visual indication of the property (e.g., color change, etc.). The sensor may be (i) located within the device 2140 and/or (ii) provided with another device or container into which the liquids and/or discharge collected by the device 2140 can be released. Alternatively, or additionally, the liquids and/or discharge collected by the device 2140 may be kept and provided to a medical provider or tested by the user. External hardware may be used to test the vaginal fluid and/or discharge. For example, an indicator strip(s) and/or nano-technology may be used for diagnostic and/or treatment purposes, by indicating, e.g., potential infections and/or imbalances.

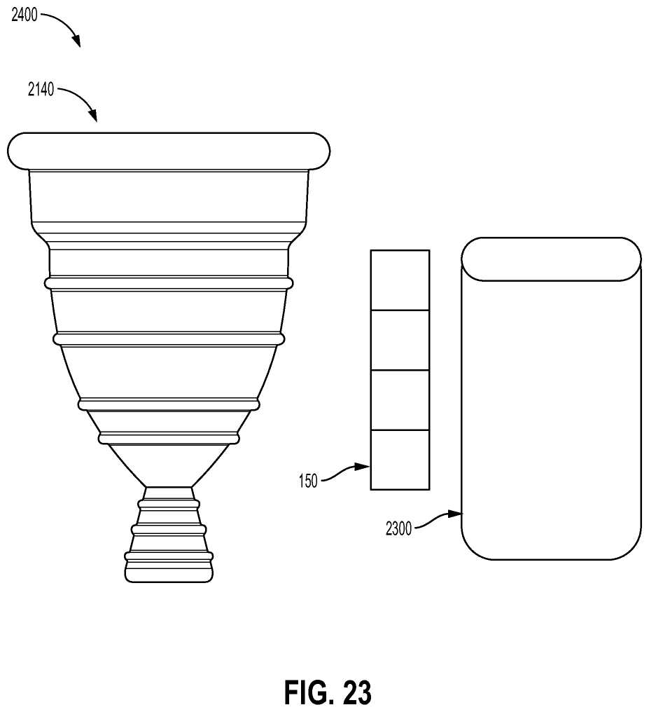

For example, as shown in the embodiment of FIG. 21B, the device 2140 includes an indicator strip 150 within the hollow interior 2152 of the upper portion 2142. This indicator strip 150 can be any form of detection and/or diagnosis strip that provides a visual indication of a certain detected disease state and/or medical condition. For example, if the pH of the vagina is outside of the norm, a color indicator may appear on a portion of the indicator strip 150. Or, if bacteria is in the vagina, the indicator strip 150 may react to the bacteria to provide a visual indication that the bacteria is present. According to one embodiment, the user can view the results of the indicator strip 150 by removing the device 40 from the vagina. The indicator strip 150 may be a sensor or other diagnostic device. Alternatively, the indicator strip 150 (or another type of diagnostic device) may be provided with a separate device and/or container (see, e.g., container 2300 of FIG. 23) into which the liquids and/or discharge collected by the device 2140 can be released for testing of such liquids and/or discharge. For example, as shown in FIG. 23, the vaginal insert device 2140, a separate device and/or container 2300, and the indicator strip 150 may be provided together as a kit 2400. Although the indicator strip 150 is shown as a separate device located within an interior space of the vaginal insert device 2140, the indicator strip 150 may take other forms. For example, the indicator strip 150 may be embedded within a wall of the vaginal insert device 2140, within one or more perforations, within one or more ridges, may be formed of the material itself, etc., or combinations thereof.

In still further embodiments, the vaginal insert device 2140 may also include (or alternatively include) a microchip 2163 (FIG. 21B) that may be responsive to vaginal pH or the presence of a bacteria when inserted. In some embodiments, the microchip 2163 may be in communication with a computer program or application that may evaluate the output from the microchip 2163. For example, the pH of the vagina or presence of a bacteria may be sent to a program or application. The program or application may evaluate the output from the microchip 2163 and may provide that information to a computer, cell phone or tablet, application, or other program, which may share the information with the user or a medical provider, or may evaluate that information to determine potential diagnosis or remedial measures that may be taken to abate symptoms or treat a condition. Alternatively, the microchip 2163 (or another type of sensing device) may be provided with a separate device and/or container (see, e.g., container 2300 of FIG. 23) into which the liquids and/or discharge collected by the device 2140 can be released for testing of such liquids and/or discharge. For example, as shown in FIG. 23, the vaginal insert device 2140, a separate device and/or container 2300, and the microchip (not shown) may be provided together as a kit 2400.

According to one embodiment, the indicator strip 150 and/or microchip 2163 can provide further diagnostic capabilities, such as, e.g., determining hormone levels (including those relating to pregnancy), ovulation levels, pH levels, temperature, pelvic floor or muscle strength, and/or an early detection component for health benefits. The indicator strip 150 and/or microchip 2163 can also provide detection and/or diagnostic capabilities relating to the detection of various STDs, such as, e.g., chlamydia, gonorrhea, HIV, syphilis, hepatitis, human papillomavirus (HPV), genital herpes, trichomoniasis, Gardnerella, mycoplasma, and/or ureaplasma. The indicator strip 150 and/or microchip 2163 can also provide detection and/or diagnostic capabilities relating to the detection of other various diseases and/or medical conditions, including, for example, yeast infections or other infections not sexually transmitted, abnormal cells, interstitial cystitis, assisting in managing interstitial cystitis, and/or any other abnormality that may be indicated by vaginal fluid, discharge and/or tissue samples (e.g., cervical tissue).

In one embodiment, the indicator strip 150 and/or microchip 2163 can be attachable/releasable or fixed to the interior of the device 2140. As shown in the embodiment of FIG. 21B, the indicator strip 150 is provided near the bottom of the upper portion 2142 of the device 2140 in order for the indicator strip to be in contact with any collected fluid and/or discharge. Although shown near the bottom interior of the vaginal insert device 2140, the indicator strip 150 may be positioned along a portion or all of the inner surface 600 of the device 2140, along a portion or all of the outer surface 602, near the upper portion, near the rim, within one or more ridges, within a rib (e.g., rib 380 of FIG. 13A), a portion of the device in contact with the vaginal walls, or any combination thereof. The indicator strip 150 may be located anywhere in which the indicator strip 150 will be in contact with vaginal tissue, vaginal fluid, collected samples (e.g., collected fluid or tissued) and/or discharge. As shown in the embodiment of FIG. 21B, the microchip 2163 is provided along one of the inner surface 600 of the device 2140. However, as with the indicator strip 150, the microchip 2163 can be alternatively provided at the bottom of the upper portion 2142 or anywhere in which the microchip 2163 will be in contact with collected fluid and/or discharge. Alternatively, as discussed above, the indicator strip 150 and/or microchip 2163 (or other types of diagnostic and/or sensing devices) may be provided with a separate device and/or container (see, e.g., container 2300 of FIG. 23) into which the liquids and/or discharge collected by the device 2140 can be released for testing of such liquids and/or discharge. For example, as shown in FIG. 23, the vaginal insert device 2140, a separate device and/or container 2300, and the indicator strip 150 and/or microchip 2163 (not shown) may be provided together as a kit 2400 to the user.

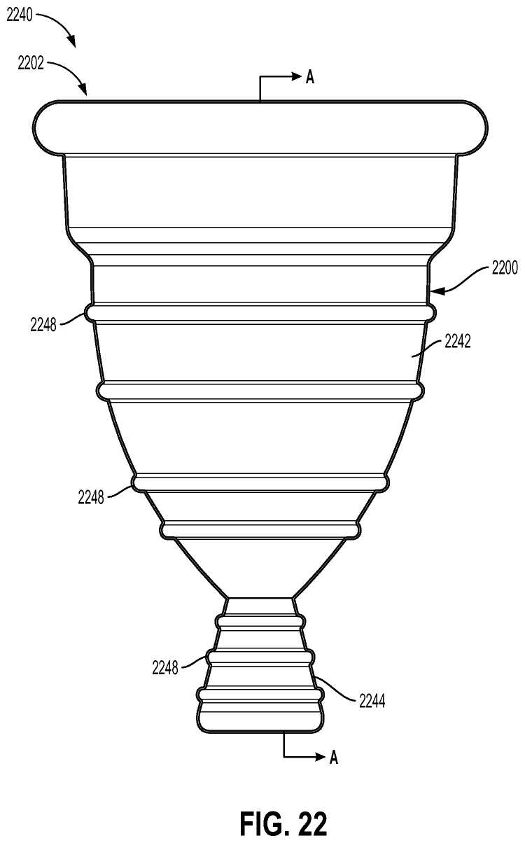

In another aspect of the disclosure, a vaginal insert device can include a texture 2200 along on at least one surface of the device 2240 as shown in FIG. 22. The texture 2200 may allow for collection of a tissue and/or fluid sample (e.g., cervical tissue) from the vaginal walls when the device 2240 is inserted and/or removed. For example, the device 2240 may include a texturized surface and/or may be dipped and/or coated in a substance to provide the texture 2200 to the exterior surface of the device 2240. The texture 2200 may be medication(s), composition(s), reagent(s), formulation(s), and/or substance(s), as described herein. For example, the texture 2200 may release medication and/or absorb vaginal discharge. In some examples, the texture 2200 may include reagents to react, diagnose, and/or provide indications, as described herein. The texture 2200 can comprise one or more different types of texture. In one embodiment, the texture 2200 is only provided to the upper portion 2242 of the device 2240. In another embodiment, the texture 2200 is provided to both the upper portion 2242 and the lower portion 2244 of the device 2240. In these embodiments, the device may or may not be hollow. The device 2240 may collect internal fluids in the same manner as the device 2140. In some examples, the device 2240 may be solid (i.e., not hollow), as in the embodiment of FIG. 22, the texture 2200 can also be provided along the top surface 2202 of the device 2240. Further, in these embodiments, the device walls may or may not be permeable. The device may have a smooth inner surface and/or a ribbed or textured outer surface, such as described herein. According to one embodiment, the texture 2200 is provided in combination with the ridges 2248 along at least a portion of the exterior of the body of the device 2240. Alternatively, the texture 2200 can be provided along at least a portion of the exterior of the body of the device 2240, without the ridges 2248 being included, or vice versa. For example, as shown in the embodiment of FIG. 22, both the ridges 2248 and the texture 2200 are provided along the upper portion 2242 of the device 2240, while only the ridges 2248 are provided along the lower portion 2244 of the device 2240.

The vaginal insert devices of FIGS. 21A, 21B, and 22 may include one or more cavities such as described with respect to FIGS. 16A, 16B and 17. The device shown and described in FIGS. 21A, 21B, and 22, as well as the other devices described herein, may be coated in medication by dipping or spraying. The device may be configured and/or manufactured to heat or cool for pain relief, for example. The device may include medication in an internal hollow portion. The medication may include time release medication that is in the silicone which is done during the liquid injection molding process. The device may include one or more types of medications. The one or more medications may include medications located in the perforations, on the surface, in the cavity, inside the cone, and/or a combination of those locations of the device.

The vaginal insert devices of the present disclosure may allow for collection of vagina discharge. For example, an indicator strip (e.g., 150) may be located inside of the vaginal insert device and/or on an exterior surface of the vaginal insert device and/or on a separate base. When located on a separate base, the vaginal insert device may be inverted and placed on the separate base upside down (e.g., with the open upper end contacting an indicator strip on the separate base). The collected sample/discharge may be transferred to the separate base and/or may be detected by the indicator strip. This indicator strip and/or separate base may then detect or indicate is abnormalities, sexually transmitted infections, bacterial vaginosis, yeast infections, urinary tract infections, etc., or any combination thereof.

The vaginal insert device of the present disclosure may collect blood from a menstrual cycle and perform a diagnostic thereto, such as, for example, with an indicator strip on or in the device and/or on a separate base. When collecting tissue and/or fluid samples, including menstrual fluids, the vaginal insert device may collect the samples without creating a seal with the internal vaginal wall. In this case, a user may wear an additional menstrual collection, such as, for example, a liner as the menstrual fluids may leak past the sides of the vaginal insert device.

The vaginal insert device of the present disclosure may provide pain management to a user (e.g., after childbirth, pre- or during menstruation, or other vaginal pain). For example, the vaginal insert device may be a hollow device that has gel inside (e.g., within a cavity as described herein). The vaginal insert device may then be cooled or frozen (e.g., by placing in a freezer or a refrigerator) prior to insertion. The cooled or frozen gel may provide pain relief for the user when inserted. Additionally, or alternatively, a coating of pain medication may be provided on the vaginal insert device and/or the pain medication may be provided for secretion in accordance with the present disclosure.

The vaginal insert device of the present disclosure may move organs within the user. In some examples, if the vaginal insert device is inserted and then clitoral stimulation is applied, the vaginal insert device in conjunction with the clitoral stimulation may strengthen pelvic floor muscles, improve tissue laxity, internal skin laxity, rejuvenate vaginal health, improve dryness, improve sexual function, increase natural lubrication, etc.

The vaginal insert device of the present disclosure may be provided as a therapeutic option for patients who have had bulking agents to the urethra and/or muscle grafting.

The vaginal insert device of the present disclosure may include a light that may sense or detect changes in tissue when inserted into the user. The vaginal insert device of the present disclosure may employ the diagnostic system described herein to determine and advise the user on the current progression of the menstrual cycle (e.g., the menstrual phase, the follicular phase, the ovulation phase, and/or the luteal phase) and/or may indicate ovulation and/or may indicate pregnancy and/or may determine fertility.

The vaginal insert device of the present disclosure may include nanotechnology, sensors, temperature sensors, diagnostic opportunities, as described herein, that may communicate or be capable of communicating with a computing device (e.g., a mobile phone, table, and/or computer, etc.).

In one aspect of the disclosure vaginal insert device may be used with a yeast infection medication. For example, in an embodiment, the vaginal insert device may be dipped in or sprayed with the yeast infection medication. Preferably, it would cover at least the outer surface of the device that comes into contact with the tissue. In other embodiments the medication may be injected into the vaginal insert device. The device that is dipped in yeast infection medication may have properties that eliminates a yeast infection in 12 hours. In other embodiments the yeast infection medication may treat or eliminate a yeast infection or the symptoms of a yeast infection in a longer or shorter time. The device may be dipped into yeast infection medication that is in a liquid, powder, or gel form. In a preferred embodiment, an ointment or medication, such as a yeast infection medication, may be a cream that is sprayed onto or injected into the device or that the vaginal insert device may be dipped into.

In some embodiments the top of the vaginal insert device may be sealed. In these embodiments the seal may dissolve when the device is inserted into the vagina. In embodiments having perforations, the perforations may additionally or alternatively have a seal around the perforations that dissolves when the device is inserted into the vagina. The seal may dissolve in response to liquid, heat, and/or other substances that may be naturally occurring within the vagina. The seal may to inhibit the yeast infection medication from escaping the device during insertion. The medication may dry or solidify onto the device or it may remain in the liquid, powder, or gel form after the device is dipped. The device may be dipped in multiple substances in some embodiments, the substances may have different topical or medicinal properties, or one substance may be used to help dry, solidify, or preserve the topical or medicinal substance.

The vaginal insert devices described herein may be collapsible for insertion and expandable once positioned in place. The vaginal insert devices may or may not be configured to support organs or prolapse when used. For example, vaginal insert devices may place pressure to improve symptoms associated with urinary incontinence but in other embodiments it may simply be a device for administering the medicinal substance.

As discussed above, the substance may have topically advantageous properties or may provide medicinally advantageous properties when applied to or absorbed by vaginal tissue. For example, the substance may include a medication intended to treat or abate the symptoms of sexually transmitted diseases, yeast infections, and/or viral or bacterial infections. The substance may also include afterbirth medications such as topical anesthesia for pain. In some embodiments, the substance may provide birth control, hormone replacement therapy, and/or cancer medication. The substance may be a prescribed or over-the-counter remedy or include essential oils or natural medications. The substance may also include a heating or cooling agent to relieve discomfort or facilitate the absorption or effectiveness of other medicinal agents in the substance. The substance used with the device may include any combination of the substances disclosed herein or other know medicinal or topical substances.

The insert device can be used to collect vaginal fluid, blood, discharge etc. The collected fluids may be used for detection or diagnosis purposes. The vaginal insert device may be further equipped with a sensor that may detect the properties of the vaginal fluid, such as PH value or the presence of a bacteria or microbe, which may be communicated to an external monitoring device. For example, an application or a medical provider system. This may be done by receptors located within the device or the liquids or discharge collected by the device may be kept and provided to a medical provider or tested by the user. External hardware may be used to test the vaginal fluid. For example, an indicator strip may indicate potential infections or imbalances. For example, if the pH of the vagina is outside of the norm, a color indicator may appear on a strip of the device. Or, if bacteria is in the vagina, the indicator strip may react to the bacteria to provide a visual indication that the bacteria is present. In still further embodiments, the vaginal insert device may include a microchip that may be responsive to vaginal pH or the presence of a bacteria when inserted. In some embodiments, the microchip may be in communication with a computer program or application that may evaluate the output from the microchip. For example, the pH of the vagina or presence of a bacteria may be sent to a program or application. The program or application may evaluate the output from the chip and may provide that information to a computer or program which may share the information with the user or medical provider, or may evaluate that information to determine potential diagnosis or remedial measures that may be taken to abate symptoms or treat a condition.

The vaginal insert devices of the present disclosure may manage, improve, prevent, treat, and/or eliminate female incontinence, including urinary incontinence, fecal incontinence, POP, or POP and urinary and/or fecal incontinence, and combinations thereof. The vaginal insert devices may not require a prescription (although may for drug or hormone delivery or as an indicator for diagnostic assistance). The vaginal insert devices may be non-implantable (though could be implantable), non-absorbent, over the counter, convenient, flexible, comfortable, and easy for a user to insert and remove, with no or minimal physician intervention. In some instances, such a vaginal insert device may be reusable. Alternatively, or additionally, the vaginal insert device may be disposable. The vaginal insert devices may eliminate concern of Toxic Shock Syndrome (TSS) by not consisting of an absorbency element which could breed bacteria, cause infection, and/or produce odor.

The vaginal insert devices of the present disclosure may prevent movement or further movement of the organs and hold the organs in place. The rim, body (e.g., cone shape), ridges, and/or rib, or combinations thereof, may work together to place pressure on the urethral sphincter, bladder neck, or bowel and prevent displacement of organs. The vaginal insert devices described herein may place pressure on the urethral sphincter, bladder neck, or bowel. The device may be adjustable so that the device is comfortable for the user to wear. The device may be adjusted by movement of the stem. Over time, the device being in place may reduce the probability of further prolapse when the user is active, for example during running, walking, jumping jacks, etc.

According to an embodiment, the present disclosure provides a method for applying pressure or force to a location of the vaginal wall of a user, the method comprising inserting a vaginal insert device into the vagina of the user, and orienting a force- or pressure-providing portion of the vaginal insert device so as to align the force- or pressure-providing portion with—or be adjacent to—the location of the vaginal wall of the user. In certain embodiments, the location of the vaginal wall is proximal to the urethral sphincter, bladder neck, or bowel. In some embodiments, applying pressure or force to the location of the vaginal wall provides pelvic organ support to the user. In certain instances, the vaginal insert device includes a rib or one or more rib sections or members as described herein, and the force- or pressure-providing portion of the vaginal insert device corresponds to the location(s) that the rib or one or more rib sections or members joins an interior wall of the device. In some embodiments, the vaginal insert device includes a rim (e.g., a circular rim, in plan view, as described herein), and the force- or pressure-providing portion of the vaginal insert device corresponds to one or more portions of the rim that provide greater pressure or force than other portions of the rim. In an embodiment, the vaginal insert device includes a stem, as described herein, which is aligned with the force- or pressure-providing portion of the vaginal insert device. In certain instances, orienting the force- or pressure-providing portion of the device includes rotating the device, such as by rotating the stem. In an embodiment, the vaginal insert device of the present disclosure may include components or features that align the device with the pelvic floor or the device itself may align the pelvic floor.

Although, the devices of the present disclosure are described and depicted as being inserted into the vagina to reduce or eliminate, for example, urinary incontinence, the device of the present disclosure may also be used to treat fecal incontinence and/or the involuntary leakage of fecal matter. The vaginal insert devices of the present disclosure may be placed in the vagina such that the device places pressure on a bowel or colon wall. The device may be rotated to adjust pressure on the bowel or colon wall as previously described. Where a rib is included in the vaginal insert device, the rib may provide pressure on the rectum and/or bowel or colon wall to effectively reduce manage, treat, prevent and/or eliminate fecal incontinence. Thus, the vaginal insert device may be placed in the vagina to place pressure on the rectum to prevent fecal incontinence or a prolapsed rectum or to prevent further displacement of the rectum. The insert may also be placed in the anus to prevent, manage, reduce, treat and/or eliminate fecal incontinence. Additional uses are also contemplated, such as, for example, menstrual uses, collection of vaginal fluids or discharge, diagnostics, exercising or strengthening muscles, secreting medicines, or hormones, monitoring of the vagina or other organs or body parts, etc., or combinations thereof.

The vaginal insert devices of the present disclosure may have multiple functions. The primary function of the device, as previously described, may be (i) to support the urethral sphincter, bladder neck, or bowel to reduce prevent, treat and/or eliminate incontinence or prevent or support prolapsed organs, (ii) to secret medications or treatments, be (iii) to collect a vaginal fluid, discharge and/or tissue sample and/or (iv) to detect and/or diagnose various diseases and/or medical conditions. Additionally, although the device may be an over-the-counter device, it may be impregnated with medication such as prescription or non-prescription homeopathic remedies or substances to address conditions for hormone levels, sexually transmitted diseases yeast infections, birth control, spermicide, as well as other substances with benefits such as antifungal or anti-infection substances that retard the growth of bacteria, fungus or diseases. The device may secrete the substance or medication. The device may be provided for hormone replacement therapy. As such, the device may be capable of drug-delivery to the vaginal cavity, for example drug delivery with a controlled, prolonged, or extended release profile. The drug delivery may be achieved by releasing drug from the outside or inside surfaces of the device and may be made from one or more than one materials or components that may be detachable or attachable. The device may also include diagnostic capabilities, such as by including an attachable/releasable or fixed indicator strip that may determine hormone levels, ovulation levels, pH levels, pelvic floor, or muscle strength or as an early detection component for health benefits. The device may have a detachable component for use during the menstrual cycle or after or before sexual activity.

The device of the present disclosure may also have a detachable component used for exercising the pelvic floor, such as weights, pellets, stainless steel pellets, indicator strip(s), a pressure sensor, nanotechnology, or other additions described herein. The weights may be added or removed or may be made of a weighted material. For example, the weights may exercise the pelvic floor muscles during exercise or Kegels or simply while the end user goes about their day the weight may force them to hold the device in place thus exercising the muscles. When additional features or components are included in the vaginal insert device of the present disclosure they may be placed into the mold prior to injection molding (or other manufacturing) such that they are integral with the device and/or they may be inserted with the material during injection molding (or other manufacturing) to be integral with the device. Alternatively, the additional features or components may be secured to the device by post processing, such as fastening, gluing, adhering, bonding, etc. to the device, walls of the device, indentations or recesses formed in the device specifically for the component, etc. Although the embodiments may be non-prescription, parts of the device or embodiments of the device may be for drug delivery or to use as an indicator for diagnosis of diseases and conditions.

The vaginal insert devices of the present disclosure may be formed of a material that provides a visual indication of diagnosis. For example, the material may change colors based on a medical condition, vitamin level, number of uses of the device (e.g., to indicate a life of the device and/or to indicate a need to change or replace the device), and/or based on the conditions of the vaginal environment (e.g., pH levels) or on the condition of the device itself. In some examples, the device is formed of a material, coated in material, embedded with a material, etc. The material may be a reagent that reacts with the sample to provide the visual indication. That is, a user may be able to remove the device and, based on the color, understanding that it is time for the device to be cleaned, recharged, replaced, and/or disposed of. A user may also remove the device and it may include diagnosing capabilities that indicate that the user is ovulating, has a yeast infection, sexually transmitted disease, certain hormone levels (e.g., an unhealthy hormone level) and/or an infection, etc.

The vaginal insert devices of the present disclosure may be implanted with a chip that communicates with Bluetooth, Wi-Fi capabilities, radio wave, microwave, or other technologies. Thus, a user may be able to communicate with a mobile device, such as phone or tablet. For example, the device may communicate a pH level or other indication of use that a user knows it is time to clean, recharge, replace, and/or dispose of the device. The device may communicate a number of pelvic floor exercise performed and be used to track pelvic floor strength. The device may communicate an environment within the vagina and be used to diagnose disease. The device may come with an app for mobile or other technology to monitor ovulation or urethral sphincter strength or pelvic floor strength. The device may be used with Wi-Fi. Additionally, if the device is worn during exercise, the device may hold the organs in place and prevent further prolapse due to long, hard constant movement such as running.

The vaginal insert devices of the present disclosure may be fabricated, such as by a molding process (e.g., liquid injection molding), with a material. The material may be an elastic and non-absorbent material, for example, a biocompatible elastomer such as medical grade silicone. The material may be a medical grade material, such as, for example a medical grade silicone. The material may be 100% medical grade silicone. Examples of silicone material suitable for use in the present device may be NuSil Technology's MED-4950 product, which is characterized as a liquid silicone rubber, Momentive LIM 6030, or Momentive LIM 6040. Alternative materials and methods of forming are contemplated. For example, the vaginal insert device may be formed of cotton, such as 100% cotton, may be any plastic, thermoplastic, polymer, elastomer. The material may have sufficient structure to apply pressure to a vaginal wall but may be flexible enough to allow for folding or compression during insertion and removal. The vaginal insert device may be compression molded, extruded, 3-d printed, rotationally molding, machined, caste, etc. The device may be made of one material. However, some embodiments may be made of multiple parts and/or from one different material or a combination of different materials and may become detachable or attachable. The device may be disassembled and reassembled for cleaning, sterilization, impregnation of drug, weights or an indication strip or various technologies such as a Bluetooth chip, radio wave, microwave, or Wi-Fi connectivity capabilities.

In an exemplary embodiment, the vaginal insert devices of the present disclosure may be used during exercise or other activity. For example, the vaginal insert device may be inserted into the body in the aforementioned manner prior to (e.g., 5 to 60 minutes before) walking, running, strength training, cardiovascular activity, kickboxing, or other high intensity activity, etc. The vaginal insert device may hold or support the pelvic organs, bladder, and/or rectum during the activity. The vaginal insert device may prevent, eliminate, or inhibit prolapse or displacement of the organs during the activity. A user may insert the vaginal insert device into the body prior to performing the activity. The activity may cause stress or place pressure on the pelvic organs, bladder, and/or rectum. In the absence of the device, the stress or pressure associated with the activity may cause the pelvic organs, bladder, and/or rectum to displace and/or prolapse and may cause discomfort to the user during activity. The vaginal insert device may support the pelvic organs, bladder, and/or rectum such as to counteract, support, inhibit, or eliminate the discomfort, stress, or pressure caused by the activity onto the organs. A user may use the vaginal insert device only during activities or during both sedentary (e.g., sleeping, resting, sitting, etc.) and active times. In an embodiment, the user may use one size of the device during activities, and may then use another size of the device during less active or sedentary times. For example, the user may use a larger size of the device during activities, and may use a smaller size of the device during less active or sedentary times.

The vaginal insert devices of the present disclosure may capture or collect vaginal fluid, discharge and/or tissue samples (e.g., cervical tissue). The vaginal fluid and/or discharge may be evaluated by the device, by releasing the collected sample onto another device (e.g., a container, indicator strip, etc.), and/or after removal by a clinician. The vaginal fluid and/or discharge may be used to diagnose various diseases and/or medical conditions, including, for example, sexually transmitted diseases, if a woman is pregnant or ovulating, hormone levels, temperature, yeast infections or other infections not sexually transmitted, abnormal cells, interstitial cystitis, assisting in managing interstitial cystitis, and/or any other abnormality that may be indicated by vaginal fluid, discharge and/or tissue samples (e.g., cervical tissue). Contrary to a menstrual cup, intended to collect all menstrual fluids, the device may collect a sample of discharge for the diagnosis and evaluation.

The removal portions of the present disclosure may be made of multiple parts and one, two, or a compound of materials which may become detachable, attachable, retractable, and expandable. The stem may rotate and/or click in place to adjust the tension or size of the device. The stem may be used for re-orienting the device. It may have markings on the stem that protrude from it, correlating with the placement of the device or strength of the device such as an arrow or numbering system. It may have a hole in it to be used as an indicator or alongside a drying rack. The vaginal insert device may increase or decrease pressure as the stem is turned. The vaginal insert device may include attachable and/or interchangeable weights. The vaginal insert may include a strip or other attachment that may determine ovulation, pH levels, etc.

In an embodiment, the rim may include one or more portions that provide greater pressure or force than other portions. The one or more portions providing greater pressure or force may be thicker, more reinforced, or composed of materials with an increased hardness (e.g., harder silicone) than the other portions of the rim. The one or more portions of the rim that provide greater pressure or force may be aligned with the stem (much like the rib or rib sections or members may be aligned with the stem, as described herein) such that the user may be able to orient the one or more portions of the rim that provide greater pressure or force with a location adjacent to the vaginal wall that provides pelvic organ support, such as a location proximal to the urethral sphincter, bladder neck, or bowel.

Although vaginal insert devices of the present disclosure are depicted herein as including one or more hollow portions in the interior of the device (e.g., between rib sections or members), in other embodiments, vaginal insert devices of the present disclosure do not include such hollow portions but are solid or semi-solid, for example by being filled by the same material(s) as that of the vaginal insert device (e.g., silicone of the same or differing hardness) or by one or more different materials (e.g., foam or gel). A vaginal insert device that is solid or semi-solid may still be squeezed or deformed, as described herein, for easier insertion of the device, and the device may then resume its original shape after insertion. In one embodiment, the vaginal insert device includes a rib, which may be divided into rib sections or members to provide support as described herein, and a filling material in the hollow portions between the rib sections or members. In an alternate embodiment, the vaginal insert device does not include a rib and includes a filling material in the hollow portion. In some instances of such an alternate embodiment, the device includes a rim having one or more portions that provide greater pressure or force than other portions, as described herein. In an embodiment, the upper portion may be closed, covered or otherwise blocked such that the inside of the device (either the hollow portions, solid portions, and/or semi solid-portions) are not exposed to atmosphere and/or to the inside of the vagina when inserted.

In accordance with the teachings of the present disclosure, the disadvantages and problems associated with known pessaries may be substantially reduced or eliminated. In accordance with the principles of the invention, a user-friendly solution is provided.

In accordance with the teachings of the present disclosure, the disadvantages and problems associated with known sample collection and/or disease testing may be substantially reduced or eliminated. In accordance with the principles of the invention, a user-friendly solution is provided.

The various devices described herein may be used with urethral sphincter injections to help exercise muscles.

In accordance with these and other embodiments of the present disclosure, a vaginal insert device may include one or more ventilation openings (e.g., holes, slits, gaps, or apertures). A ventilation opening may be located at the point where the lower end of the upper portion and a stem intersect. One or more ventilation openings may be located in the wall or the rim on the upper portion. The ventilation opening may be used as a position indicator, for ventilation, or for both a position indicator and ventilation.

In accordance with the disclosure, the device may be configured for use for pain relief with or without medication. For example, the device may be configured to have different temperatures. Heat and/or cold are contemplated depending upon the indication being addressed. The temperature may vary depending upon the intended use. For example, if used for pain relief either with or without medication, an example may be to chill and/or freeze the device. The lower, cold, or frozen temperature of the device in use may help treat the problem and/or the symptoms of the problem. The device may be configured or constructed to accommodate the different temperatures, including, for example, the device may include liquid, gel, and/or features that would allow it to be frozen and/or retain cold during use. Or the material that the device is made of may have properties for temperature control. The various devices may include ridges that may or may not contain medication for treatment of various issues as well as may or may not include gel, liquid and/or powder for pain relief. The device may be heated or cooled for relief of vaginal scarring or tearing due to childbirth. This temperature feature may be used alone or in combination with various treatments and/or a steroid, for example.

In accordance with these and other embodiments of the present disclosure, a vaginal insert device may include an applicator used during insertion and/or removal of the device. In some embodiments, the applicator contains at least the upper portion when it is in a more compact shape. The applicator may attach and detach from the device. The applicator may work as an indicator for positioning, expanding, and/or retracting the device. The applicator may have indicators for positioning such as a ridge, arrow, bump, protrusion, lettering, or numbering system. The applicator may have indentions or holes to indicate position. The applicator and device may click into place or otherwise fasten together.

The present disclosure describes various embodiments of a vaginal insert device and the method of using the vaginal insert device. The vaginal insert devices of the present disclosure collect a sample, while also allowing for detecting and/or diagnosing various diseases and/or medical conditions. The vaginal insert device of the present disclosure may not require a prescription, may be non-absorbent, over the counter, convenient, comfortable, and easy for a user to insert and remove, with no or minimal physician intervention. Such a vaginal insert device may be reusable but may also be disposable. Various users are contemplated and users may include patients, consumer, etc.

Medical and other advantages of the present disclosure may be readily apparent to one skilled in the art from the figures, description and claims included herein. The objects and advantages of the embodiments will be realized and achieved at least by the elements, features, and combinations particularly pointed out in the claims.

All examples and conditional language recited herein are intended for pedagogical objects to aid the reader in understanding the disclosure and the concepts contributed by the inventor to furthering the art, and are construed as being without limitation to such specifically recited examples and conditions. Although embodiments of the present disclosure have been described in detail, it should be understood that various changes, substitutions, and alterations could be made hereto without departing from the spirit and scope of the disclosure

1-103. (canceled)

104. A vaginal insert device configured for placement in a vagina, the vaginal insert device comprising:
a cone-shaped body having a hollow interior; and
a sensor configured to detect a property of a vaginal fluid or vaginal tissue.

105. The vaginal insert device of claim 104, wherein the sensor is a reagent, an indicator strip, an electrical sensor, a receptor, or nano-technology.

106. The vaginal insert device of claim 104, wherein the property is a pH value, a presence of a bacteria, a presence of a microbe, a vitamin level, an ovulation level, a hormone level, a temperature, a pelvic floor or muscle strength, a sexually transmitted disease, a yeast infection, a nonsexually transmitted infection, abnormal cells, interstitial cystitis, or an abnormality in vaginal fluid or tissue.

107. The vaginal insert device of claim 104, further comprising a microchip configured to communicate the property to a user or a medical professional.

108. The vaginal insert device of claim 107, wherein the microchip is configured to communicate via Bluetooth®, Wi-Fi, or radio waves such that the property is communicated to a mobile device or computer.

109. The vaginal insert device of claim 104, wherein the sensor is located within the hollow interior of the cone-shaped body.

110. The vaginal insert device of claim 104, wherein the sensor provides a visual indication of the property.

111. The vaginal insert device of claim 110, wherein the sensor is an indicator strip and the visual indication is a change in color of the indicator strip.

112. The vaginal insert device of claim 104, wherein the sensor is an indicator strip that is a separate device from the cone-shaped body and is located within the hollow interior.

113. The vaginal insert device of claim 104, wherein the sensor is an indicator strip embedded within a wall of the vaginal insert device.

114. The vaginal insert device of claim 104, wherein the sensor is an indicator strip embedded within one or more perforations in the cone-shaped body.

115. The vaginal insert device of claim 104, wherein the sensor is an indicator strip embedded within one or more ridges in the cone-shaped body.

116. The vaginal insert device of claim 104, wherein the sensor is an indicator strip releasably connected to the hollow interior.

117. The vaginal insert device of claim 104, wherein the sensor is an indicator strip provided near a bottom of the cone-shaped body.

118. The vaginal insert device of claim 104, wherein the sensor is an indicator strip is positioned along a portion or all of an inner surface of the cone-shaped body, along a portion or all of an outer surface of the cone-shaped body, near an upper portion of the cone-shaped body, near a rim of the cone-shaped body, within one or more ridges of the cone-shaped body, within a rib within the cone-shaped body, within a portion of the vaginal insert device in contact with the vaginal walls, or any combination there.

119. A system for detecting a property of a vaginal fluid or vaginal tissue, the system comprising:
a vaginal insert device having a cone-shaped body, the vaginal insert device configured to collect the vaginal fluid or vaginal tissue;
a container configured to receive the vaginal fluid or vaginal tissue; and
an indicator strip, a microchip configured to detect the property of the vaginal fluid or vaginal tissue in the container.

120. The system of claim 119, wherein the property is a pH value, a presence of a bacteria, a presence of a microbe, a vitamin level, an ovulation level, a hormone level, a temperature, a pelvic floor or muscle strength, a sexually transmitted disease, a yeast infection, a nonsexually transmitted infection, abnormal cells, interstitial cystitis, or an abnormality in vaginal fluid or tissue.

121. A method for detecting a property in a vagina, the method comprising
providing a vaginal insert device having a cone-shaped body;
providing a sensor;
detecting the property with the sensor; and
communicating the property to a user or medical provider of the vaginal insert device.

122. The method of claim 121, wherein providing the sensor comprises coupling or embedding the sensor to the vaginal insert device.

123. The method of claim 121, wherein providing the sensor comprises providing the sensor in a container separate from the vaginal insert device.

124. The method of claim 121, wherein communicating the property to the user or medical provider comprises communicating the property or a diagnosis based on the property to an external monitoring device.

125. The method of claim 121, wherein communicating the property to the user or medical provider comprises providing a visual indication of the property.

126. The method of claim 125, wherein the visual indication is a change in color of the vaginal insert device.

127. The method of claim 121, wherein the sensor is a reagent, an indicator strip, an electrical sensor, a receptor, or nano-technology.

128. The method of claim 121, wherein the property is a pH value, a presence of a bacteria, a presence of a microbe, a vitamin level, an ovulation level, a hormone level, a temperature, a pelvic floor or muscle strength, a sexually transmitted disease, a yeast infection, a nonsexually transmitted infection, abnormal cells, interstitial cystitis, or an abnormality in vaginal fluid or tissue.

129. The method of claim 121, wherein communicating the property further comprises communicate the property to the user or medical provider with a microchip.

130. The method of claim 121, further comprising providing the sensor within a hollow interior of the cone-shaped body.

131. The method of claim 121, wherein the sensor is an indicator strip that is a separate device from the cone-shaped body and is located within the hollow interior.

132. The method of claim 121, wherein the sensor is an indicator strip embedded within a wall of the vaginal insert device, embedded within one or more perforations in the cone-shaped body, embedded within one or more ridges in the cone-shaped body, releasably connected to the hollow interior, or provided near a bottom of the cone-shaped body.

133. The method of claim 121, wherein the sensor is an indicator strip is positioned along a portion or all of an inner surface of the cone-shaped body, along a portion or all of an outer surface of the cone-shaped body, near an upper portion of the cone-shaped body, near a rim of the cone-shaped body, within one or more ridges of the cone-shaped body, within a rib within the cone-shaped body, within a portion of the vaginal insert device in contact with the vaginal walls, or any combination there.
